(19) United States
(12) Patent Application Publication
(10) Pub. No.: US 2010/0032293 A1
(43) Pub. Date: Feb. 11, 2010

(54) Droplet Dispensing Device and Methods

(75) Inventors: Michael G. Pollack, Durham, NC (US); Vamsee K. Pamula, Durham, NC (US); Vijay Srinivasan, Durham, NC (US)

Correspondence Address: ADVANCED LIQUID LOGIC, INC.;C/O WARD AND SMITH, P.A., 1001 COLLEGE COURT, P.O. BOX 867, NEW BERN, NC, 28563-0867, US

(73) Assignee: ADVANCED LIQUID LOGIC, INC., Research Triangle Park, NC (US)

(21) Appl. No.: 12/531,809

(22) PCT Filed: Apr. 10, 2008
(86) PCT No.: PCT/US08/59955

§ 371 Date: Oct. 15, 2009

Related U.S. Application Data
(60) Provisional application No. 60/910,897, filed on Apr. 10, 2007.
(60) Provisional application No. 60/980,202, filed on Oct. 16, 2007.

(51) Int. Cl. C07K 1/26; G01N 27/00

(52) U.S. Cl. 204/450; 204/600

(57) ABSTRACT

The invention provides nonlimiting examples of structures for and methods of dispensing droplets in a droplet actuator. The droplet actuator structures and methods of the invention exhibit numerous advantages over droplet actuators of the prior art. In various embodiments, the structures and methods of the invention provide, among other things, improved efficiency, throughput, scalability, and/or droplet uniformity, as compared with existing droplet actuators. Further, in some embodiments, the droplet actuators provide configurations for improved methods of loading and/or unloading fluid and/or droplets. In yet other embodiments, the droplet actuators provide fluid loading configurations for loading numerous fluid reservoirs in a substantially simultaneous and/or substantially sequential manner.

### 2 RELATED PATENT APPLICATIONS

This application claims priority to U.S. Patent Application No. 60/910,897, filed on Apr. 10, 2007, entitled “Droplet dispensing methods for droplet microactuators”; and U.S. Patent Application No. 60/980,202, filed on Oct. 17, 2007, entitled “Droplet dispensing designs and methods for droplet actuators”; the entire disclosures of which are incorporated herein by reference.

### 1 GOVERNMENT INTEREST

This invention was made with government support under DK066956-02 awarded by the National Institutes of Health of the United States. The United States Government has certain rights in the invention.

### 3 BACKGROUND

Droplet actuators are used to conduct a wide variety of droplet operations. A droplet actuator typically includes a substrate associated with electrodes configured for conducting droplet operations on a droplet operations surface thereof and may also include a second substrate arranged in a generally parallel fashion in relation to the droplet operations surface to form a gap in which droplet operations are effected. The gap is typically filled with a filler fluid that is immiscible with the fluid that is to be subjected to droplet operations on the droplet actuator. Among the droplet operations which may be effected on a droplet actuator is the dispensing of a droplet from a fluid source. There is need in the art for improved approaches to dispensing droplets on a droplet actuator.

### 4 BRIEF DESCRIPTION OF THE INVENTION

The invention provides a method of forming multiple droplets on a droplet actuator. The method may, for example, involve providing a droplet actuator. Various basic droplet actuator structures are described herein and/or are known in the art. These may be modified as described herein to perform the unique methods of the invention. In one embodiment, the modified droplet of the invention includes a base substrate having: (i) droplet operation electrodes configured for conducting one or more droplet operations; (ii) a perimeter barrier surrounding the electrodes comprising multiple openings, each opening approximately adjacent to one or more electrodes of the droplet operation electrodes; and (iii) a flow path exterior to the perimeter barrier and arranged to flow fluid through the multiple openings into proximity with the one or more electrodes. Droplets may be dispensed by flowing fluid through the flow path, through the openings in the perimeter barrier and into proximity with the one or more electrodes and conducting one or more droplet operations to form droplets on the droplet operation electrodes.

In another embodiment, the method of forming multiple droplets on a droplet actuator, includes providing fluid on one or more activated electrodes and draining fluid from around the activated electrodes, leaving droplets on the activated droplet operation electrodes. Fluid may, for example, be provided on activated electrodes by (i) flowing fluid onto at least a portion of the droplet operation electrodes; and (ii) activating one or more of the droplet operation electrodes.

Another embodiment relates to a method of dispensing one or more sub-droplets from a droplet on a droplet actuator, the method including: (i) providing a path of electrodes in proximity to a droplet; (ii) activating electrodes in the path of electrodes to form the droplet into a slug arranged along the path of electrodes and transport the slug along the path of electrodes; and (iii) selectively deactivating electrodes in the path of electrodes at a trailing end of the slug to pinch off one or more sub-droplets from the trailing end of the slug.

Yet another embodiment relates to a method of dispensing one or more sub-droplets from a droplet on a droplet actuator, the method: (i) providing a path of electrodes in proximity to a droplet; (b) activating electrodes in the path of electrodes to form the droplet into a slug arranged along the path of electrodes and transport the slug along the path of electrodes; and (c) selectively deactivating electrodes in the path of electrodes at a trailing end of the slug to pinch off one or more sub-droplets from the trailing end of the slug.

In another aspect, the method of dispensing one or more sub-droplets from a droplet on a droplet actuator makes use of a droplet actuator comprising: (i) a base substrate comprising electrodes configured for conducting droplet operations; and (ii) a top substrate separated from the base substrate to form a gap, the top plate comprising: (1) a reservoir; and (2) an opening forming a fluid path from the reservoir into the gap. The reservoir opening may be arranged such that when a fluid is provided in the reservoir, the fluid is brought into proximity to a first electrode, which first electrode is adjacent to a second electrode. The method may include (a) causing the first and second electrodes to be activated, thereby causing fluid to flow from the reservoir onto the first and second electrodes; and (b) deactivating the first electrode, causing a droplet to form on the second electrode and causing the remaining fluid to return substantially to the reservoir.

The invention also provides method of dispensing one or more sub-droplets from a droplet on a droplet actuator including a base substrate with a droplet operation electrodes configured for conducting droplet operations and a recessed reservoir region configured for holding a droplet in proximity to one or more of the electrodes. The droplet actuator may also include a top substrate separated from the base substrate to form a gap. The method may include (a) causing a first electrode adjacent to the recessed reservoir region and a second electrode adjacent to the first electrode to be activated, thereby causing fluid to flow from the reservoir onto the first and second electrodes; and (b) deactivating the first electrode, causing a droplet to form on the second electrode and causing the remaining fluid to return substantially to the recessed reservoir region.

In another aspect, the invention provides a method of dispensing one or more sub-droplets from a droplet on a droplet actuator having a set of electrodes with a set of successively smaller substantially crescent shaped planar electrodes, arranged concentrically substantially in a common plane along a common axis positioned midway between vertices of the substantially crescent-shaped electrodes, wherein each successively smaller electrode is positioned adjacent to the next larger electrode. The droplet actuator may also include a set of planar dispensing electrodes substantially in a common plane with the crescent shaped electrodes, arranged substantially along the common axis of the crescent. In some cases, the droplet actuator includes a top substrate separated from the base substrate to form a gap. The method generally involves (a) causing a first electrode adjacent to the recessed reservoir region and a second electrode adjacent to the first electrode to be activated, thereby causing fluid to flow from the reservoir onto the first and second electrodes; and (c) deactivating the first electrode (or an electrode intermediate to the crescent shaped electrodes and the terminal activated electrode or electrodes), causing a droplet to form on the second electrode and causing the remaining fluid to return substantially to the recessed reservoir region.

A further aspect of the invention is a droplet actuator having a base substrate with (a) droplet operation electrodes configured for conducting one or more droplet operations; (b) a perimeter barrier surrounding the electrodes comprising multiple openings, each opening approximately adjacent to one or more electrodes of the droplet operation electrodes; and (c) a flow path formed in the perimeter barrier and arranged to flow fluid through the multiple openings into proximity with the one or more electrodes.

Another droplet actuator of the invention includes (a) a base substrate having electrodes configured for conducting droplet operations; and (b) a top substrate separated from the base substrate to form a gap, the top plate comprising: (i) a reservoir; and (ii) an opening forming a fluid path from the reservoir into the gap; wherein the reservoir opening is arranged such that when a fluid is provided in the reservoir, the fluid is brought into proximity to a first one of the electrodes.

Still another aspect relates to a droplet actuator with (a) a base substrate comprising: (i) droplet operation electrodes configured for conducting droplet operations; and (ii) a recessed reservoir region configured for holding a droplet in proximity to one or more of the droplet operation electrodes; and (b) a top substrate separated from the base substrate to form a gap.

A further droplet actuator embodiment includes a set of electrodes comprising a set of successively smaller substantially crescent shaped planar electrodes, arranged: concentrically; or substantially in a common plane along a common axis positioned midway between vertices of the substantially crescent-shaped electrodes, wherein each successively smaller electrode is positioned adjacent to the next larger electrode.

In another method aspect, the invention provides a method of manipulating a droplet on a droplet actuator, the method comprising: (a) providing a droplet actuator comprising: (i) a reservoir electrode comprising an array of multiple, independently controllable electrodes; (ii) a structure proximate the reservoir electrode comprising an opening; (iii) a transfer electrode positioned in fluid communication with both the reservoir electrode and the opening; and (iv) a flow path through the opening, transfer electrode and the reservoir electrode; and (b) flowing fluid through the flow path.

Another method of the invention relates to forming a droplet on a droplet actuator, the method comprising: (a) providing a droplet actuator comprising: (i) a reservoir electrode; (ii) a structure proximate the reservoir electrode comprising an opening; (iii) a transfer electrode positioned in fluid communication with both the reservoir electrode and the opening, wherein the transfer electrode at least partially overlaps with the opening; and (iv) a flow path through the opening and transfer electrode and the reservoir electrode; and (b) flowing fluid through the flow path.

Yet another method of manipulating a droplet on a droplet actuator according to the invention includes (a) providing a droplet actuator comprising: (i) a droplet operation electrode configured for conducting one or more droplet operations; (ii) a structure comprising an opening; and (iii) a reservoir electrode proximate both the droplet operation electrode and the opening; and (b) providing a flow path through the opening, reservoir electrode and droplet operation electrode.

The invention also provides a method of manipulating a droplet on a droplet actuator, the method including the following steps: (a) supplying a droplet to a reservoir electrode; (b) embedding an electrode within the reservoir electrode; (c) selectively activating electrodes in a path of electrodes that includes the embedded electrode to form the droplet into a slug arranged along the path of electrodes and to transport the slug along the path of electrodes; and (d) selectively deactivating electrodes in the path of electrodes at a trailing end of the slug to pinch off one or more sub-droplets from the trailing end of the slug.

In still another aspect, the method of manipulating droplets on a droplet actuator includes: (a) providing a droplet actuator comprising: (i) a reservoir electrode; (ii) a structure proximate the reservoir electrode comprising an opening; (iii) a plurality of electrode arrays respectively in fluid communication with the reservoir electrode; and (iv) a plurality of flow paths through the opening, reservoir electrode and each respective electrode array; and (b) flowing fluid through at least one of the flow paths.

The invention also provides a method of manipulating droplets on a droplet actuator, the method comprising: (a) providing a droplet actuator comprising a structure comprising an opening in fluid connection with a plurality of flow paths; and (b) flowing fluid through the plurality of flow paths.

In another aspect, the invention provides method of manipulating droplets on a droplet actuator, the method comprising: (a) providing a droplet actuator comprising: (i) a structure comprising an opening in fluid connection with a plurality of other openings; (ii) a plurality of fluid reservoirs respectively in fluid communication with each of the other openings; (iii) a plurality of electrodes in respective fluid communication with the fluid reservoirs; and (iv) a plurality of flow paths through the opening, the other openings, the reservoirs and the electrodes; and (b) flowing fluid through the plurality of flow paths.

The invention provides a method of manipulating a droplet on a droplet actuator, the method comprising: (a) supplying a droplet to a reservoir electrode; (b) embedding an electrode within the reservoir electrode; (c) selectively activating the embedded electrode so as to retain a portion of the droplet proximate the embedded electrode; and (d) evacuating another portion of the droplet from the reservoir electrode.

Another method of dispersing magnetic beads within a droplet in a droplet actuator includes: (a) providing a droplet actuator, comprising: (i) a plurality of transport electrodes configured to transport the droplet; and (ii) a magnet field present at a portion of the plurality of transport electrodes; (b) transporting the droplet along the plurality of transport electrodes away from the magnetic field; and (c) transporting the droplet along the plurality of transport electrodes towards the magnetic field.

The invention provides a method of manipulating a droplet comprising magnetic beads within a droplet actuator, the method comprising: (a) providing a droplet actuator, comprising: (i) a plurality of transport electrodes configured to transport the droplet; and (ii) a magnetic field present at a portion of the plurality of transport electrodes; and (b) positioning a magnetic shielding material in the droplet actuator to selectively minimize the magnetic field.

The invention also provides a method of re-suspending particulate within a droplet in a droplet actuator, the method comprising: (a) providing a droplet actuator, comprising: (i) a plurality of independently controllable reservoir electrodes configured to manipulate a droplet; and (ii) a plurality of transport electrodes in fluid communication with the plurality of reservoir electrodes; and (b) independently operating the plurality of reservoir electrodes to cause the particulate to re-suspend within the droplet.

The invention provides a method of re-suspending particulate within a droplet in a droplet actuator, the method comprising: (a) providing a droplet actuator, comprising: (i) a reservoir electrode configured to manipulate a droplet; and (ii) a plurality of transport electrodes in fluid communication with the reservoir electrode; (b) separating a slug of the droplet from the droplet on the reservoir electrode; and (c) recombining the slug with the droplet at the reservoir electrode.

Moreover, the invention provides a method of re-suspending particulate within a droplet in a droplet actuator, the method comprising: (a) providing a droplet actuator, comprising: (i) a reservoir electrode configured to manipulate a droplet; and (ii) a plurality of transport electrodes in fluid communication with the reservoir electrode; and (b) selectively applying across the reservoir electrode a voltage from an alternating current source to agitate the droplet.

In another aspect, the invention provides a method of manipulating a droplet comprising magnetic beads within a droplet actuator, the method comprising: (a) providing a droplet actuator, comprising: (i) a plurality of transport electrodes configured to transport the droplet; and (ii) a magnetic field present at a portion of the plurality of transport electrodes; and (b) positioning a plurality of magnets so as to selectively minimize the magnetic field.

In yet another aspect, the invention provides a method of dispensing magnetic beads within a droplet on a droplet actuator, the method comprising: (a) providing a droplet actuator, comprising: (i) top and bottom plates; (ii) a plurality of magnetic fields respectively present proximate the top and bottom plates, wherein at least one of the magnet fields is selectively alterable; and (iii) a plurality of transport electrodes positioned along at least one of the top and bottom surfaces; (b) positioning the droplet between the top and bottom surfaces; and (c) selectively altering at least one of the magnetic fields.

The invention also provides a method of splitting a droplet comprising a magnetic bead in a droplet actuator, the method comprising: (a) providing a droplet actuator comprising: (i) a plurality of transport electrodes configured to transport the droplet; and (ii) a magnetic field present at the plurality of transport electrodes; (b) immobilizing the magnetic bead using the magnetic field; and (c) using the plurality of transport electrodes to split the droplet into first and second droplets, wherein the magnetic bead remains substantially immobilized.

Further, the invention provides a method of splitting a droplet comprising a magnetic bead in a droplet actuator, the method comprising: (a) providing a droplet actuator comprising: (i) a plurality of transport electrodes configured to transport the droplet, the plurality including an elongated electrode having a length at least twice that of a transport electrode of the plurality; and (b) splitting the droplet using the elongated electrode.

The invention also provides a method of splitting a droplet comprising a magnetic bead in a droplet actuator, the method comprising: (a) providing a droplet actuator comprising: (i) a plurality of transport electrodes configured to transport the droplet, the plurality including a segmented electrode having at least one of a column and row of segments; and (b) splitting the droplet using the segmented electrode.

Further, the invention provides a method of detecting a component of supernatant, the method comprising: (a) removing excess unbound antibody from a plurality of beads; (b) adding a chemiluminescent substrate to the beads; and (c) detecting the component of the supernatant.

These and other aspects of the invention will be apparent from the ensuing description and claims.

### 5 DEFINITIONS

As used herein, the following terms have the meanings indicated.

“Activate” with reference to one or more electrodes means effecting a change in the electrical state of the one or more electrodes which results in a droplet operation.

“Bead,” with respect to beads on a droplet actuator, means any bead or particle that is capable of interacting with a droplet on or in proximity with a droplet actuator. Beads may be any of a wide variety of shapes, such as spherical, generally spherical, egg shaped, disc shaped, cubical and other three dimensional shapes. The bead may, for example, be capable of being transported in a droplet on a droplet actuator or otherwise configured with respect to a droplet actuator in a manner which permits a droplet on the droplet actuator to be brought into contact with the bead, on the droplet actuator and/or off the droplet actuator. Beads may be manufactured using a wide variety of materials, including for example, resins, and polymers. The beads may be any suitable size, including for example, microbeads, microparticles, nanobeads and nanoparticles. In some cases, beads are magnetically responsive; in other cases beads are not significantly magnetically responsive. For magnetically responsive beads, the magnetically responsive material may constitute substantially all of a bead or one component only of a bead. The remainder of the bead may include, among other things, polymeric material, coatings, and moieties which permit attachment of an assay reagent. Examples of suitable magnetically responsive beads are described in U.S. Patent Publication No. 2005-0260686, entitled, “Multiplex flow assays preferably with magnetic particles as solid phase,” published on Nov. 24, 2005, the entire disclosure of which is incorporated herein by reference for its teaching concerning magnetically responsive materials and beads. The beads may include one or more populations of biological cells adhered thereto. In some cases, the biological cells are a substantially pure population. In other cases, the biological cells include different cell populations, e.g., cell populations which interact with one another.

“Dispense,” “dispensing” and the like means a droplet operation in which a droplet is formed from a larger volume of fluid. In some embodiments, the droplet is formed atop an electrode on a droplet operations substrate. The larger volume of fluid may, for example, be a continuous fluid source, a relatively large volume of fluid extending into a fluid path and/or reservoir associated with a droplet actuator, or a source droplet associated with a droplet actuator surface. The larger volume of fluid may be loaded on a droplet actuator, partially loaded on a droplet actuator, or otherwise associated with a droplet actuator in sufficient proximity with an electrode to effect a dispensing operation.

“Droplet” means a volume of liquid on a droplet actuator that is at least partially bounded by filler fluid. For example, a droplet may be completely surrounded by filler fluid or may be bounded by filler fluid and one or more surfaces of the droplet actuator. Droplets may take a wide variety of shapes; nonlimiting examples include generally disc shaped, slug shaped, truncated sphere, ellipsoid, spherical, partially compressed sphere, hemispherical, ovoid, cylindrical, and various shapes formed during droplet operations, such as merging or splitting or formed as a result of contact of such shapes with one or more surfaces of a droplet actuator.

“Droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to size of the resulting droplets (i.e., the size of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading.

“Immobilize” with respect to magnetically responsive beads, means that the beads are substantially restrained in position in a droplet or in filler fluid on a droplet actuator. For example, in one embodiment, immobilized beads are sufficiently restrained in position to permit execution of a splitting operation on a droplet, yielding one droplet with substantially all of the beads and one droplet substantially lacking in the beads.

“Magnetically responsive” means responsive to a magnetic field. “Magnetically responsive beads” include or are composed of magnetically responsive materials. Examples of magnetically responsive materials include paramagnetic materials, ferromagnetic materials, ferrimagnetic materials, and metamagnetic materials. Examples of suitable paramagnetic materials include iron, nickel, and cobalt, as well as metal oxides, such as Fe₃O₄, BaFe₁₂O₁₉, CoO, NiO, Mn₂O₃, Cr₂O₃, and CoMnP.

“Washing” with respect to washing a magnetically responsive bead means reducing the amount and/or concentration of one or more substances in contact with the magnetically responsive bead or exposed to the magnetically responsive bead from a droplet in contact with the magnetically responsive bead. The reduction in the amount and/or concentration of the substance may be partial, substantially complete, or even complete. The substance may be any of a wide variety of substances; examples include target substances for further analysis, and unwanted substances, such as components of a sample, contaminants, and/or excess reagent. In some embodiments, a washing operation begins with a starting droplet in contact with a magnetically responsive bead, where the droplet includes an initial amount and initial concentration of a substance. The washing operation may proceed using a variety of droplet operations. The washing operation may yield a droplet including the magnetically responsive bead, where the droplet has a total amount and/or concentration of the substance which is less than the initial amount and/or concentration of the substance. Other embodiments are described elsewhere herein, and still others will be immediately apparent in view of the present disclosure.

The terms “top” and “bottom” are used throughout the description with reference to the top and bottom substrates of the droplet actuator for convenience only, since the droplet actuator is functional regardless of its position in space.

When a given component, such as a layer, region or substrate, is referred to herein as being disposed or formed “on” another component, that given component can be directly on the other component or, alternatively, intervening components (for example, one or more coatings, layers, interlayers, electrodes or contacts) can also be present. It will be further understood that the terms “disposed on” and “formed on” are used interchangeably to describe how a given component is positioned or situated in relation to another component. Hence, the terms “disposed on” and “formed on” are not intended to introduce any limitations relating to particular methods of material transport, deposition, or fabrication.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface.

When a droplet is described as being “on” or “loaded on” a droplet actuator, it should be understood that the droplet is arranged on the droplet actuator in a manner which facilitates using the droplet actuator to conduct one or more droplet operations on the droplet, the droplet is arranged on the droplet actuator in a manner which facilitates sensing of a property of or a signal from the droplet, and/or the droplet has been subjected to a droplet operation on the droplet actuator.

Further, the terms “top” and “bottom” or “horizontal” and “vertical” are sometimes used with reference to portions of the figures. These terms are used with reference to regions of the figures and are not intended to limit the orientation in space of the actual elements of the invention.

### 6 BRIEF DESCRIPTION OF THE DRAWINGS

FIGS. 1A, 1B, and 1C show a top view of a droplet dispensing portion of a droplet actuator in which fluid is flowed through multiple openings into proximity with droplet operations electrodes;

FIGS. 2A, 2B and 2C show a top view of a droplet dispensing portion of a droplet actuator in which fluid is flowed across and/or retracted from activated electrodes to form droplets;

FIG. 3 shows a top view of a droplet dispensing portion of another embodiment of a droplet actuator in which fluid is flowed across and/or retracted from activated electrodes to form droplets;

FIGS. 4A, 4B, 4C, and 4D illustrate a top view of a droplet dispensing configuration of a portion of a droplet actuator in which droplets are transported across electrodes using droplet operations to form droplets;

FIG. 5 illustrates a top view of another droplet dispensing configuration of a portion of a droplet actuator in which droplets are transported across electrodes using droplet operations to form droplets;

FIGS. 6A, 6B, and 6C show a side view of a segment of a droplet actuator and illustrate a droplet dispensing process that forms small droplets from a large droplet by use of electrowetting, gravity forces, and capillary forces;

FIGS. 7A, 7B, and 7C show a side view of a portion of a droplet actuator in which a reduced gap height is used to facilitate dispensing of droplets;

FIG. 8 illustrates a top view of a droplet dispensing configuration of a portion of a droplet actuator for efficiently handling varying volumes of liquid in the fluid reservoir;

FIGS. 9A and 9B illustrates a top view of another droplet dispensing configuration of a portion of a droplet actuator for efficiently handling varying volumes of liquid in the fluid reservoir;

FIG. 10 illustrates a top view of yet another droplet dispensing configuration of a portion of a droplet actuator for efficiently handling varying volumes of liquid in the fluid reservoir;

FIG. 11 illustrates a top view of another droplet dispensing configuration of a portion of a droplet actuator for efficiently handling varying volumes of liquid in the fluid reservoir;

FIG. 12 illustrates a top view of yet another droplet dispensing configuration of a portion of a droplet actuator for efficiently handling varying volumes of liquid in the fluid reservoir;

FIGS. 13A, 13B, and 13C illustrate an electrode array of a droplet actuator and shows a droplet dispensing process in which droplets are dispensed diagonally in multiple directions;

FIG. 14 illustrates a top view of a reservoir droplet dispensing configuration of a droplet actuator in relation to an opening for loading\unloading fluid;

FIGS. 15A, 15B, 15C, 15D, 15E, and 15D illustrate multiple top views, respectively, of multiple example reservoir droplet dispensing configurations of a droplet actuator, shown in relation to an opening for loading and/or unloading fluid;

FIGS. 16A, 16B, and 16C illustrate multiple top views of certain example openings in relation to a fluid reservoir of a droplet actuator;

FIG. 17 illustrates a top view of a droplet dispensing configuration of a portion of a droplet actuator and illustrates a process of dispensing droplets;

FIG. 18 illustrates another view of the droplet dispensing configuration and process of dispensing droplets of FIG. 17;

FIG. 19 illustrates a top view of another droplet dispensing configuration of a portion of a droplet actuator and illustrates another process of dispensing droplets;

FIG. 20A illustrates another top view of the droplet dispensing configuration of FIG. 17 and illustrates a process of agitating droplets and/or priming the fluid reservoir in a droplet actuator;

FIG. 20B illustrates yet another top view of the droplet dispensing configuration of FIG. 17 and illustrates a process of agitating fluid in a droplet actuator;

FIG. 21A illustrates a top view of a droplet dispensing configuration of a portion of a droplet actuator and illustrates a process of disposing of a 1× size droplet in a droplet actuator;

FIG. 21B illustrates another top view of the droplet dispensing configuration of FIG. 21A and illustrates a process of disposing of a 2× size droplet in a droplet actuator;

FIG. 22A illustrates a top view of a dual-purpose droplet dispensing configuration of a portion of a droplet actuator and illustrates a process of dispensing droplets in a droplet actuator;

FIG. 22B illustrates another top view of the dual-purpose droplet dispensing configuration of FIG. 22A and illustrates a process of disposing of droplets in a droplet actuator;

FIG. 23A illustrates a top view of an example droplet dispensing configuration for dispensing droplets in multiple directions from a single reservoir in a droplet actuator;

FIG. 23B illustrates a top view of another example droplet dispensing configuration for dispensing droplets in multiple directions from a single reservoir in a droplet actuator;

FIG. 23C illustrates a top view of yet another example droplet dispensing configuration for dispensing droplets in multiple directions from a single reservoir in a droplet actuator;

FIG. 24A illustrates a top view of a portion of a droplet actuator for parallel distribution of fluid to multiple fluid reservoirs using a single opening;

FIG. 24B illustrates a cross-sectional view of the droplet actuator taken along line AA of FIG. 24A;

FIG. 25A illustrates a top view of a portion of a droplet actuator for serial distribution of fluid to multiple fluid reservoirs using a single opening;

FIG. 25B illustrates a cross-sectional view of the droplet actuator taken along line BB of FIG. 25A;

FIGS. 26A and 26B illustrate top views of an example droplet dispensing configuration of a droplet actuator that includes a droplet forming electrode that is embedded in a larger reservoir electrode; and

FIG. 26C illustrates a top view of an example droplet dispensing configuration of a droplet actuator that includes multiple droplet forming electrodes that are embedded in a larger reservoir electrode.

### 7 DESCRIPTION

The invention provides an improved droplet actuator and methods of making and using the droplet actuator. Various aspects of the invention provide enhanced droplet dispensing relative to existing droplet actuators. Enhanced droplet dispensing may, for example, include aspects which provide enhanced efficiency, throughput, scalability, and/or droplet uniformity. Other aspects provide improved unloading of droplets from a droplet actuator relative to existing droplet actuators. The various aspects of the invention described in the ensuing sections may be provided on a droplet actuator individually or in any combination with other aspects.

7.1 Droplet Dispensing Structures and Methods

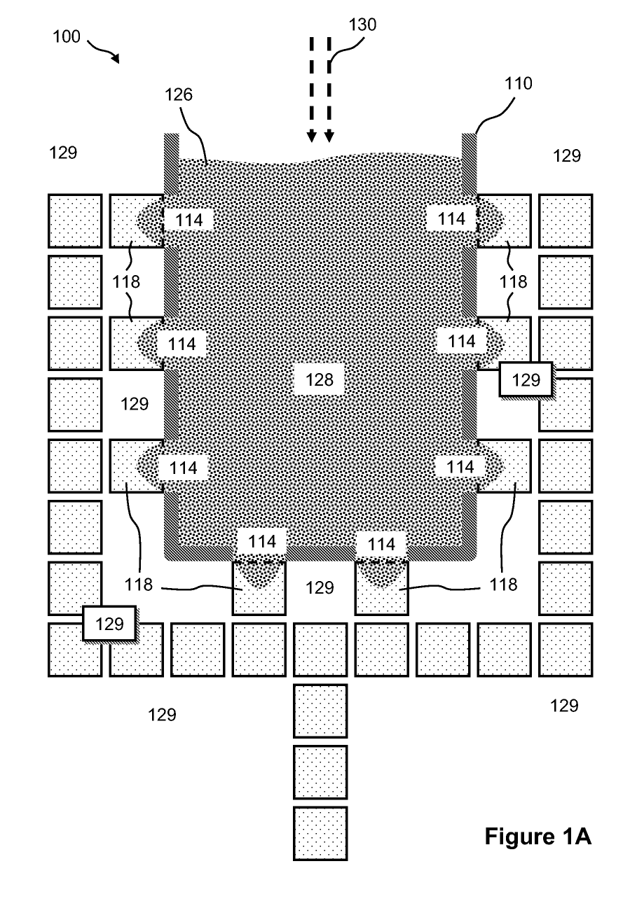

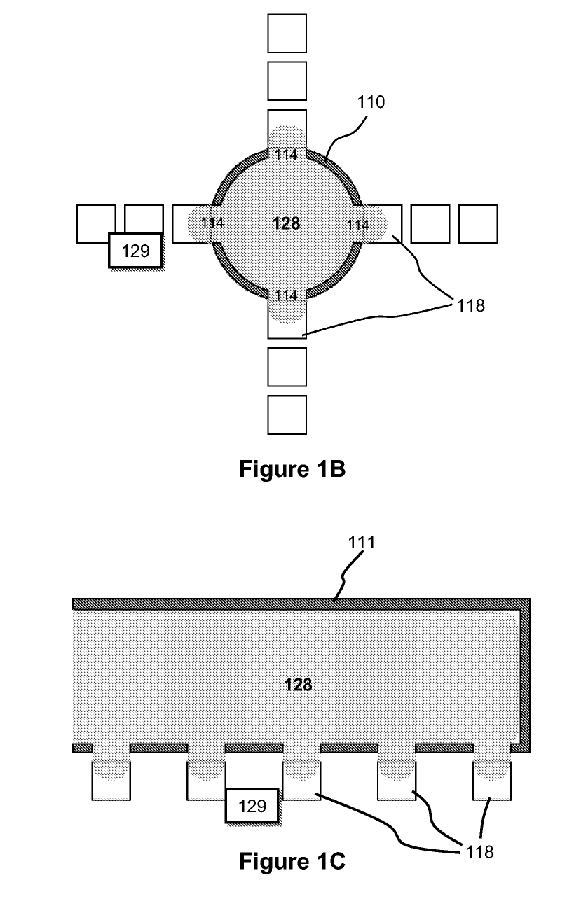

FIGS. 1A, 1B and 1C show top views of various embodiments of a region of a droplet operations surface 129 of a droplet actuator showing a droplet dispensing configuration 100. The illustrated embodiment is useful, among other things, for dispensing multiple droplets in a substantially simultaneous manner. Configuration 100 includes a fluid reservoir 128. Fluid reservoir 128 is defined by wall 110, by the substrate that forms the droplet operations surface 129 and optionally by a top substrate (not shown). It will be appreciated that any of a wide variety of configurations is possible, so long as the configuration provides a fluid path that permits liquid 126 to flow under appropriate conditions from the reservoir 128 onto the droplet operations surface 129.

Wall 110 of fluid reservoir 128 may include multiple openings 114. Each opening 114 provides a fluid path from the reservoir 128 to the droplet operations surface 129. In some embodiments, surfaces of the wall 110, the top substrate (not shown), and/or the bottom substrate 129, associated with openings 114 may be sufficiently hydrophobic in character to inhibit the flow of liquid 126 through openings 114. A hydrophobic coating, such as a Teflon® coating can be used to achieve this purpose. In other embodiments, flow may be inhibited by keeping the openings sufficiently small and/or by including physical flow barriers in proximity to the openings. The inhibition of flow may be overcome by forcing fluid into reservoir 128, e.g., using a pressure source and/or a vacuum source.

As illustrated in FIG. 1A, droplet dispensing operations may take place on three sides of fluid reservoir 128. Fluid reservoir 128 essentially projects onto a droplet operations surface 129 so that droplets may be dispensed on three sides thereof. In a dispensing operation, liquid 126 is forced through openings 114 into proximity with electrodes 118. When liquid 126 is in proximity with electrodes 118, electrodes 118 may be used to conduct droplet dispensing operations. FIG. 1B illustrates an alternative arrangement in which droplets are dispensed in multiple directions from a centrally located reservoir 128.

FIG. 1C shows another embodiment in which droplets are dispensed in parallel in a single direction from a reservoir 128.

One or more electrodes 118 may be provided in association with the droplet operations surface and/or the top substrate (when present). The electrodes 118 are configured for conducting one or more droplet operations on the droplet operations surface 129, e.g., dispensing of droplets on the droplet operations surface 129.

In operation, at a certain pressure level, liquid 126 fills fluid reservoir 128 without passing through openings 114. At a certain higher pressure level, liquid 126 flows through openings 114 into sufficient proximity with electrodes 118 to permit electrodes 118 to facilitate one or more droplet operations.

In one embodiment, when one or more of electrodes 118 is activated, liquid 126 in reservoir 128 may be retracted to leave droplets of fluid on electrodes 118. In this embodiment, pressure source 130 provides the force needed to push out and pulling back the volume of liquid 126 within fluid reservoir 128. For example, the supply of liquid 126 may be held under pressure via pressure source 130, which is a variable pressure source.

In another embodiment, additional electrodes adjacent to electrodes 118 may be activated, further extending liquid 126 onto the droplet operations surface. Intermediate electrodes, such as electrodes 118, may be deactivated to cause the formation of droplets on the additional electrodes. As illustrated by this embodiment, a change in pressure from the pressure source may not be required to facilitate droplet formation, though in some cases droplet formation may be enhanced by a change in pressure from the pressure source.

FIGS. 1B and 1C illustrate embodiments which are similar to the embodiments illustrated in FIG. 1A. As illustrated in FIG. 1B, fluid reservoir 128 may be provided within droplet operations surface so that fluid may be dispensed in multiple directions on the surface. In the specifically illustrated embodiment, droplets may be dispensed radially in four directions from a central fluid source. Another embodiment, droplets may be dispensed radially in 2, 3, 4, 5, 6, 7, 8, 9, 10, 20, 30, 40, 50 or more directions from a central fluid source. Other embodiments permit dispensing from a central fluid source, but the dispensing path is not necessarily radially oriented relative to the central fluid source. Further, as illustrated in FIG. 1C, the fluid reservoir 128 may extend alongside droplet operations surface 129 so that droplets are dispensed on one side thereof.

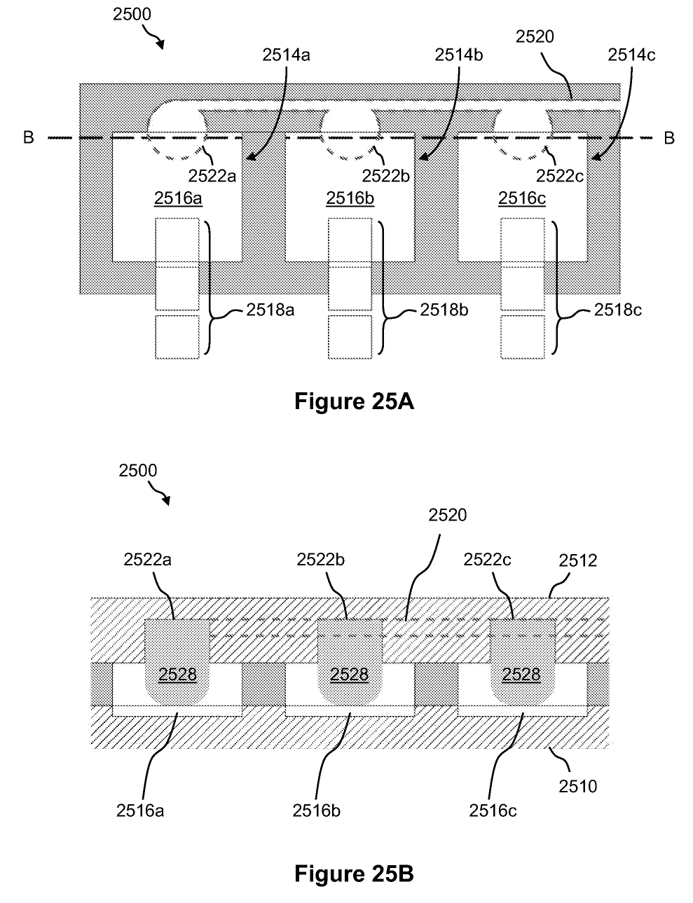

It will be appreciated that the embodiment of FIGS. 25A and 25B (discussed below) is an alternative aspect of the embodiment illustrated in FIG. 1. In FIG. 1, the reservoir 128 is oriented on generally the same plane as the droplet operations surface 129. In contrast, in FIGS. 25A and 25B, the fluid source bringing is located in a substantially different plane relative to the droplet operations surface. It should also be noted that the fluid source in FIGS. 25A and 25B may in other embodiments be located in substantially the same plane as the droplet operations surface 129.

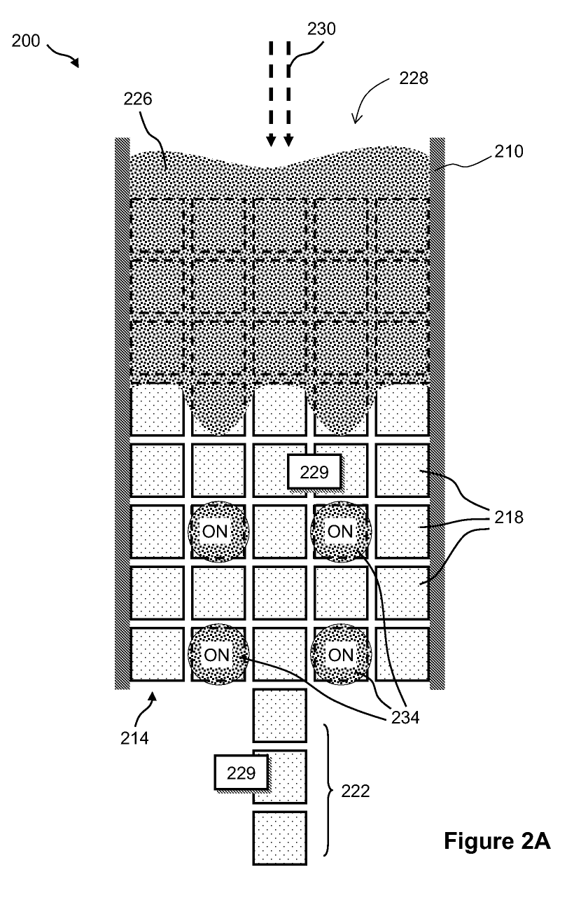

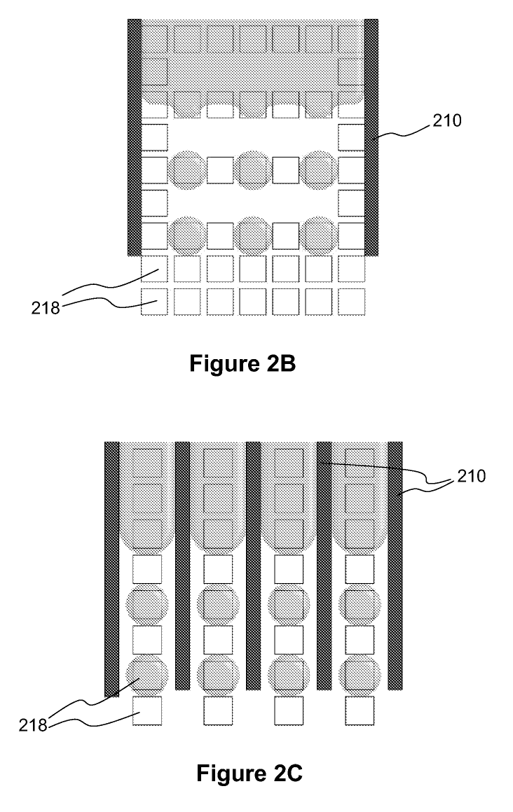

FIGS. 2A, 2B and 2C show top view of droplet dispensing configurations 200 of a portion of a droplet actuator. The illustrated embodiment is useful, among other things, for dispensing multiple droplets from a source fluid 226. The droplets may, for example, be dispensed onto a droplet operations surface 229.

As illustrated in FIG. 2A, configuration 200 includes a fluid reservoir 228, though it will be appreciated that in some cases the fluid reservoir could represent substantially the entire droplet operations surface 229. As illustrated in FIG. 2A, fluid reservoir 228 is defined by walls 210, by the substrate that forms the droplet operations surface 229 and optionally by a top substrate (not shown). A path or as illustrated here, an array 214 of electrodes 218 is associated with the droplet operations surface 229 and/or associated with the top substrate (not shown) within area of fluid reservoir 228 defined by walls 210. Other electrodes 222 may be provided outside the fluid reservoir or in some cases the fluid reservoir may take up substantially the entire droplet operations surface. Electrode array 214 is illustrated as an array of N×M electrodes, within which there may be individual control of each electrodes or of specific sets of electrodes. Of course, in alternative embodiments, paths or other patterns of electrodes will suffice, for example, see FIGS. 2B and 2C.

An arrangement of droplet operations electrodes 222 may be included, fed by electrode array 214, for conducting subsequent droplet operations using dispensed droplets 234. Droplet operations electrodes 222 may also be provided in various paths or arrays.

Fluid reservoir 228 may be filled or partially filled with a volume of liquid 226 from which droplets may be dispensed. Droplets are dispensed by providing activated electrodes within the filled region of fluid reservoir 228. When the liquid 226 is retracted, droplets remain on the activated electrodes. In the specific example illustrated, a pressure source 230 provides the force for pushing out and pulling back the volume of liquid 226 within fluid reservoir 228. For example, the pressure source 230 may be a variable pressure source. One of more pressure sources may be used as needed.

In operation, liquid 226 may be flowed into fluid reservoir 228 so that liquid 226 covers a portion of, or substantially all of, electrode array 214. Liquid 226 may then be retracted or otherwise removed from transport electrodes 222. Selected electrodes 218 may be activated prior to retracting liquid 226, so that droplets 234 are retained on the activated electrodes 218. In one embodiment, an array of electrodes, including every other electrode 218 is activated, resulting in formation of an array of droplets. The droplets are left behind on the activated electrodes 218 in the wake of the retracting or otherwise removing liquid 226. Upon formation, droplets 234 may be subjected to droplet operations using electrodes 218 and or other electrodes 222 exterior to the reservoir 228.

FIGS. 2B and 2C illustrate examples of alternative arrangements to the arrangement shown in FIG. 2A. FIG. 2B illustrates an arrangement in which electrodes 218 are provided in paths rather than in an array. FIG. 2C illustrates an arrangement in which multiple walls 218 separate individual paths of electrodes 218.

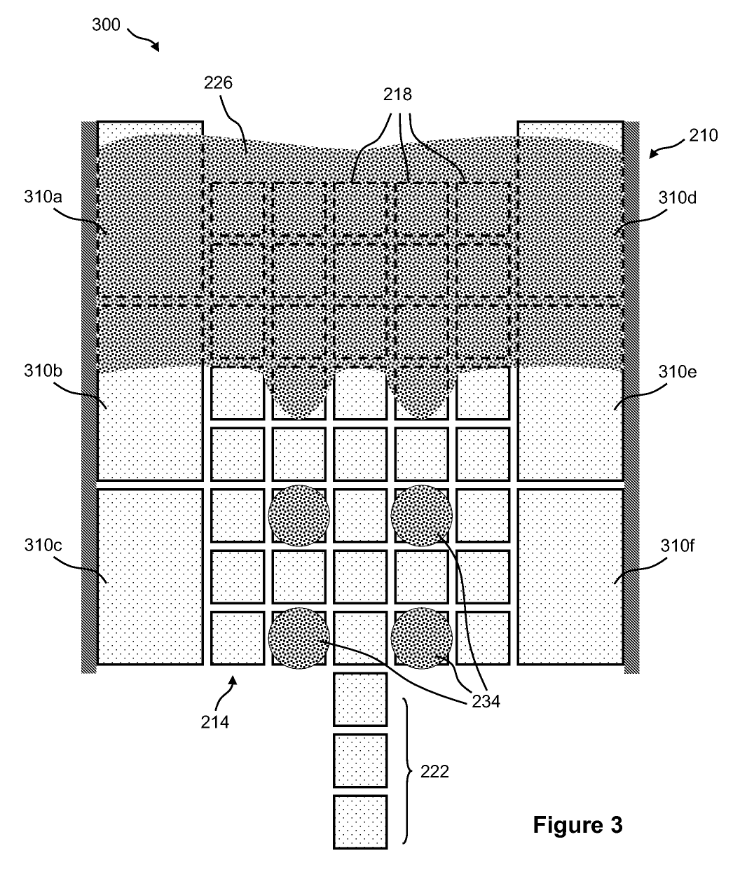

FIG. 3 illustrates a top view of a droplet dispensing configuration 300 of a portion of a droplet actuator. Droplet dispensing configuration 300 is substantially the same as droplet dispensing configuration 200 of FIG. 2, except that a pressure mechanism (e.g., pressure source 230) is replaced or supplemented with an electrowetting mechanism as the energy source for moving the volume of liquid 226 across the droplet forming electrodes 218. In the example illustrated, a series of flow electrodes 310, such as flow electrodes 310a, 310b, 310c, 310d, 310e, and 310f, are arranged at the outer edges of electrode array 214, as shown in FIG. 3. Flow electrodes 310 provide an electrowetting mechanism for moving the volume of liquid 222 across the droplet forming electrodes 218 in the process of forming droplets 234. Each electrode 310 may, for example, be several times larger, e.g., 2×, 3×, 4×, 5×, 6×, or larger, as compared to the area of a droplet operations electrode 218.

In operation, flow electrodes 310 are activated to draw liquid 226 across droplet forming electrodes 218. Certain of the droplet forming electrodes 218 are activated. Flow electrodes 310 are then deactivated, causing the liquid 226 to retract and leaving droplets 234 on the activated droplet forming electrodes.

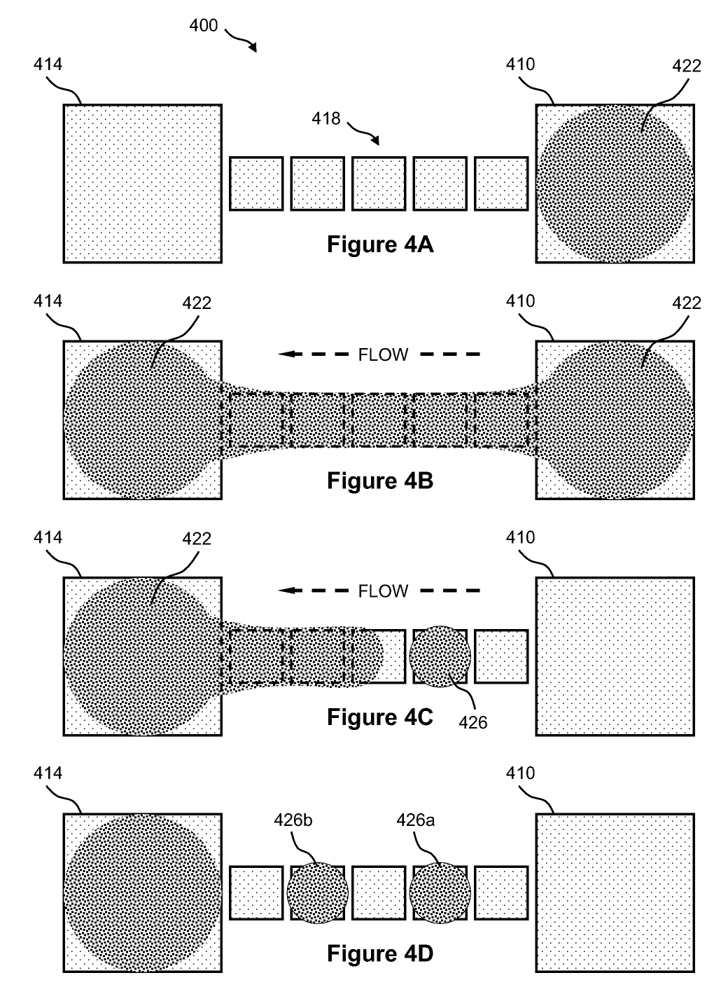

FIGS. 4A, 4B, 4C, and 4D illustrate a top view of a droplet dispensing configuration 400 of a portion of a droplet actuator and illustrate a droplet dispensing process that dispenses droplets as liquid flows in one direction (as compared to the flow in and retract schemes illustrated in FIGS. 2 and 3). Droplet dispensing configuration 400 may include a reservoir electrode 410, which may, in one embodiment, be an electrode of a source fluid reservoir. Droplet dispensing configuration 400 may also include a reservoir electrode 414, which may, in one embodiment, be an electrode of a destination fluid reservoir. Droplet dispensing configuration 400 further includes a set of transport electrodes 418 that are arranged between reservoir electrode 410 and reservoir electrode 414. In another embodiment, one or both of the reservoir electrode and the destination electrode may be replaced with one or more droplet operations electrodes, such as transport electrodes 418.

FIG. 4A shows an example of a first step of a droplet dispensing process in which reservoir electrode 410 only is activated and, thus, substantially all of the volume of a liquid 422 is present at reservoir electrode 410. Liquid 422 is the liquid from which droplets to be subjected to droplet operations may be dispensed.

FIG. 4B shows an example of a second step of the droplet dispensing process in which reservoir electrode 410 remains activated and transport electrodes 418 and reservoir electrode 414 are activated. As a result, the volume of liquid 422 extends from reservoir electrode 410, across all transport electrodes 418, and to reservoir electrode 414. In doing so, the volume of fluid that originated at reservoir electrode 410 is substantially distributed across reservoir electrode 410, transport electrodes 418, and reservoir electrode 414. Additional fluid may also be drawn into the gap from an external fluid source (not shown) associated with reservoir 422. A substantially continuous “slug” of liquid 422 is thus formed from reservoir electrode 410 to reservoir electrode 414.

FIG. 4C shows an example of a third step of the droplet dispensing process in which reservoir electrode 410 is deactivated, every other of transport electrode 418 only is activated, and reservoir electrode 414 is activated. As the slug of liquid 422 changes its footprint and moves across transport electrodes 418 and toward reservoir electrode 414, a droplet, such as a droplet 426, is left behind on each transport electrode 418 that is activated. Ideally, reservoir electrode 410 is deactivated followed sequentially by deactivation of a series of one or more of the intermediate transport electrode 418, sequentially forming droplets 426 from the trailing liquid at each of the activated electrodes.

FIG. 4D shows an example of a fourth step of the droplet dispensing process in which, after forming a certain number of droplets 426, reservoir electrode 414 remains activated and the remaining volume of liquid 422 (excluding droplets 426a and 426b) is collected at reservoir electrode 414. FIG. 4D shows, for example, a droplet 426a and a droplet 426b that are formed on certain transport electrodes 418 that are activated. Of course, a wide variety of droplet arrangements is possible, depending on which of the electrodes 418 remain activated and which are deactivated.

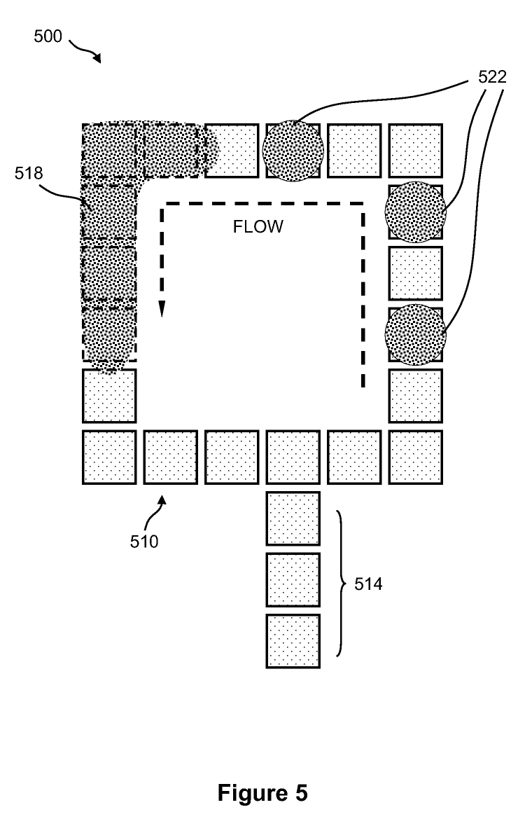

FIG. 5 illustrates a top view of another example of a droplet dispensing configuration 500 of a portion of a droplet actuator. Like the embodiment illustrated in FIG. 4, this embodiment dispenses droplets from a trailing end of a moving slug of liquid. Droplet dispensing configuration 500 may include a path of electrodes 510. As illustrated, the path is arranged in a loop, but any arrangement that forms a path along which a slug of liquid can be transported is suitable. A “slug” of liquid 518 is provided from which droplets to be subjected to droplet operations may be formed. Electrodes are activated to cause the slug of liquid 518 to be transported around the loop of electrodes 510. In the wake of the moving slug of liquid 518, certain electrodes 510, e.g., every other electrode 510, may remain activated, thereby forming droplets 522 on these certain electrodes 510, as the slug continues to be transported away from the trailing activated electrodes. In the loop embodiment, transport electrodes 514 may be used for transporting liquid 518 and droplets 522 in and out of the loop for further droplet operations.

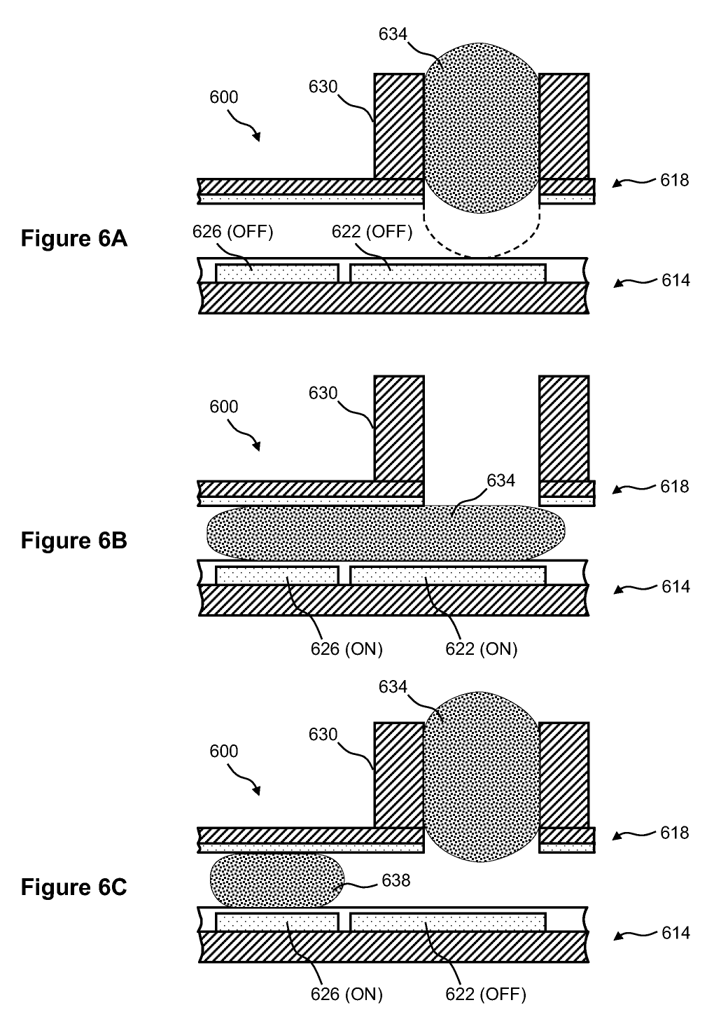

FIGS. 6A, 6B, and 6C illustrate a side view (cross-section) of a segment of a droplet actuator 600 and show a droplet dispensing process that forms small droplets from a large droplet. Droplet actuator 600 may include a bottom substrate 614 that is separated from a top substrate 618 by a gap. An electrode 622 and one or more transport electrodes 626 may be associated with bottom substrate 614. A fluid reservoir 630 or other fluid source may be associated with top substrate 618. Fluid reservoir 630 may, for example, be a well that opens to, or otherwise includes a fluid path extending to, the gap between bottom substrate 614 and top substrate 618. A droplet 634 may be contained within fluid reservoir 630, from which droplets may be dispensed.

FIG. 6A shows an example of a first step of a droplet dispensing process. Droplet 634 is substantially contained within fluid reservoir 630. Without the use electrowetting and when all electrodes are deactivated, liquid supply droplet 634 stays substantially within the well of fluid reservoir 630.

FIG. 6B shows an example of a second step of the droplet dispensing process in which electrode 622 and the adjacent transport electrode 626 are both activated in order to generate sufficient pressure difference in the gap of droplet actuator 600 to cause liquid supply droplet 634 to flow out of fluid reservoir 630 and onto electrode 622 and transport electrode 626.

FIG. 6C shows an example of a third step of the droplet dispensing process in which electrode 622 is deactivated and the adjacent transport electrode 626 remains activated. Capillary forces cause liquid supply droplet 634 to return to fluid reservoir 630, leaving a droplet 638 behind that is formed on transport electrode 626.

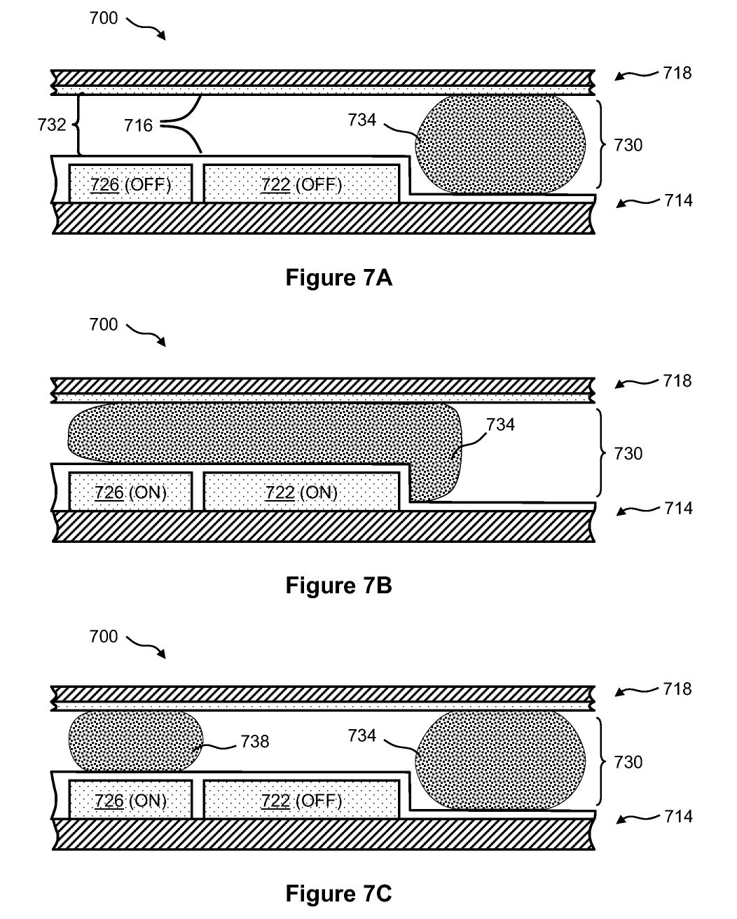

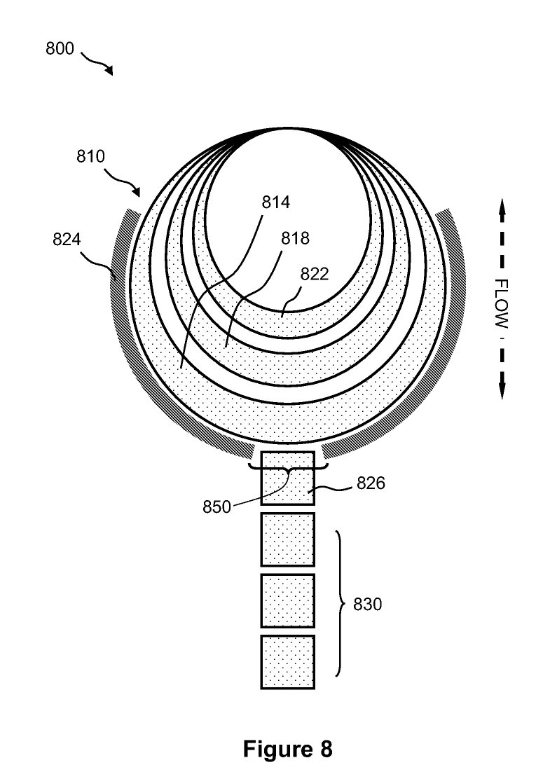

FIGS. 7A, 7B, and 7C illustrate a side view of a portion of a droplet actuator 700 and a droplet dispensing process. The droplet dispensing process forms a sub-droplet from a source droplet by making use of electrowetting in combination with other forces, such as surface tension and/or capillary forces. Droplet actuator 700 may include a bottom substrate 714 that is separated from a top substrate 718 by a gap 732. Top substrate 718 and bottom substrate 714 establish droplet operations surfaces 716, facing gap 732. An electrode 722 and one or more droplet operations electrodes, such as transport electrodes 726 may be associated with bottom substrate 714.

A fluid reservoir 730 may be formed by providing a region between top substrate 718 and bottom substrate 714 of increased gap height relative to the height of the gap 732 in the droplet operations region of the droplet actuator. In the illustrated embodiment, the gap 730 forming the fluid reservoir may be formed by features within bottom substrate 714 only, top substrate 718 only, or within the combination of bottom substrate 714 and top substrate 718. Alternatively, the fluid reservoir 730 may be formed by a separate structure that abuts the top substrate 718 and bottom substrate 714, such that the height of gap 730 is established by substrates or structures other than the top substrate 718 and bottom substrate 714. For example a reservoir or other fluid source may abut top substrate 718 and bottom substrate 714 and provide a fluid source and fluid path for supplying liquid to the droplet operations surface of the droplet actuator. A liquid supply droplet 734 may be contained within gap 730, from which droplets to be subjected to droplet operations may be dispensed. The reservoir formed by gap 730 or its alternatives may itself be coupled in fluid communication with an external liquid supply source.

FIG. 7A shows a first step of a droplet dispensing process. Liquid supply droplet 734 is provided and substantially contained within fluid reservoir 730 in proximity with electrode 722. When electrode 722 is deactivated, liquid supply droplet 734 remains substantially within fluid reservoir 730.

FIG. 7B shows an example of a second step of the droplet dispensing process. Electrode 722 and the adjacent electrode 726 are both activated in order to cause liquid supply droplet 734 to flow into gap 732 onto electrode 722 and transport electrode 726.

FIG. 7C shows an example of a third step of the droplet dispensing process. Electrode 722 is deactivated and the adjacent transport electrode 726 remains activated. A portion of liquid supply droplet 734 returns to fluid reservoir 730, leaving a droplet 738 on transport electrode 726.

FIG. 8 illustrates a top view of a droplet dispensing configuration 800 of a portion of a droplet actuator. Droplet dispensing configuration 800 includes a fluid reservoir 810 that may be formed in association with a single droplet operations substrate or between two substrates of a droplet actuator that are separated by a gap. Disposed within fluid reservoir 810 may be one or more electrodes for efficiently performing operations on the volume of liquid therein. The volume of liquid is variable. In one example, fluid reservoir 810 may include an electrode 814, an electrode 818, and an electrode 822 within the area of fluid reservoir 810. A barrier 824 may be provided to serve as a boundary of fluid reservoir 810, separating the reservoir from the remainder of the droplet operations surface. The barrier 824 includes an opening 850 through which liquid may flow into proximity with adjacent electrode 826 that feeds a set of droplet operations electrodes 830.

Electrode 814, electrode 818, and electrode 822 may be, for example, individually-controlled concentric crescent moon-shaped electrodes that are widest at the opening of fluid reservoir 810 and narrowest opposite the opening of fluid reservoir 810, as shown in FIG. 8. As illustrated, the reservoir electrodes are formed from substantially perfect circles; however, it will be appreciated that angles may be introduced, and a variety of shapes may be employed in which the electrode is thickest in proximity to electrode 826 and narrowest at a point which is generally distal to electrode 826. As the volume of liquid (not shown) within fluid reservoir 810 varies, e.g., due to the process of dispensing droplets via electrode 826 and transport electrodes 830, certain of one or more electrodes 814, 818, and 822 are activated for most efficient operations on the liquid. All three electrodes may be activated to cause larger volumes of liquid to flow into proximity with electrode 826. Reservoir electrodes 814 and 818 may be activated together for smaller volumes. Reservoir 814 may be activated alone for still smaller volumes. As a result, the volume of liquid may be moved efficiently into proximity with electrode 826. Once in proximity with electrode 826, droplet operations for dispensing subdroplets may be executed using electrode 826 and electrodes 830, e.g., by activating a row of electrodes to cause liquid to flow onto the droplet operations surface and deactivating an intermediate one or more of the electrodes to produce a subdroplet on one or more of the electrodes on the droplet operations surface.

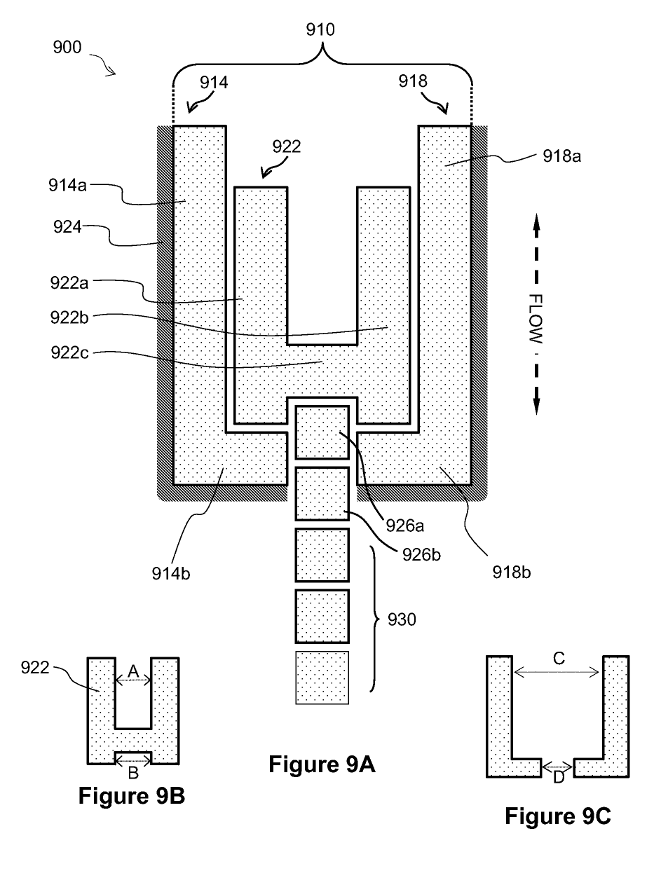

FIGS. 9A and 9B illustrates a top view of another droplet dispensing configuration 900, which is similar to the configuration 800 illustrated in FIG. 8. Droplet dispensing configuration 900 includes a fluid reservoir 910 that may be formed on a single substrate or between two substrates of a droplet actuator that are separated by a gap. One or more reservoir electrodes 922 and/or 914 are disposed within fluid reservoir 910.

In one example, fluid reservoir 910 may includes a central H-shaped reservoir electrode 922, which is also illustrated in FIG. 9B. The H-shaped electrode includes two generally parallel segments 922a/922b joined (at a point other than the end-point) by a connecting segment 922c. As illustrated, the two generally parallel segments 922a/922b are positioned generally at right angles relative to the connecting segment 922c; however, it will be appreciated that obtuse or acute angles may be employed as alternatives. The connecting segment 922c connects the two generally parallel segments 922a/922b at a point other than the end-point, two gaps A and B (see FIG. 9B) are formed, one gap A at the top and one gap B at the bottom portion of the H-shaped electrode. One or more droplet operations electrodes, such as droplet dispensing electrodes 926 may be inset into either of these gaps. In an alternative embodiment, the connecting segment 922c connects the two generally parallel segments 922a/922b at an endpoint proximal to the droplet dispensing electrodes, thereby forming a U-shaped reservoir electrode rather than an H-shaped reservoir electrode. In one embodiment, an H-shaped electrode is provided having first and second gaps (A and B) and a droplet operations electrode 924 positioned in one of the gaps. The droplet dispensing electrodes 926 may be associated with additional droplet operations electrodes 930 configured for conducting droplet operations using dispensed droplets.

Fluid reservoir 910 may also include two L-shaped electrodes 914 and 918. One of the L-shaped electrodes 918 may be reflected along a vertical axis, i.e., it may be a mirror image of an “L.” Each of the L-shaped electrodes 914 and 918 includes an elongated segment 914a/918a and a shorter segment 914a/914b. The elongated segments 914a/918a may in some embodiments be placed at a right angle relative to the corresponding shorter segments 914a/914b. The two L-shaped electrodes may be electrically coupled to one another such that they function as a single electrode. An L-shaped electrode 914 and a mirror image L-shaped electrode 918 may be aligned with the horizontal segments 914b/918b facing each other and a gap D formed therebetween. This arrangement also provides a gap C between the horizontal vertical members of the L-shaped electrodes 914/918. In one embodiment, an L-shaped electrode is provided along with a mirror image of an L-shaped electrode, where the horizontal portions of the two L-shaped electrodes are aligned with each other and separated to form a gap therebetween, and a droplet operations electrode is positioned in the gap. The droplet dispensing electrodes 926 may be associated with additional droplet operations electrodes 930 configured for conducting droplet operations using dispensed droplets.

In another embodiment, an L-shaped electrode is provided along with a mirror image of an L-shaped electrode, where the horizontal portions of the two L-shaped electrodes are aligned with each other and separated to form a gap therebetween. An H-shaped electrode is provided in the gap between the vertical members of the L-shaped electrodes, such that a gap in the H-shaped electrode is generally aligned with the gap between the horizontal members of the L-shaped electrodes. A first droplet operations electrode is provided at least partially in the gap of the H-shaped electrode that is aligned with the gap between the horizontal members of the L-shaped electrodes. A second droplet operations electrode is provided at least partially in the gap formed by the horizontal members of the L-shaped electrodes.

Electrode 914, electrode 918, and electrode 922 may be, for example, individually-controlled electrodes of differing size, location, and shape, as shown in FIG. 9. In this way, as the volume of liquid (not shown) within fluid reservoir 910 varies over time, due to the process of dispensing droplets via electrode 926 and transport electrodes 930, certain of one or more electrodes 914, 918, and 922 are activated for most efficient operation on the liquid.

In operation, the H-shaped electrode 922 and L-shaped electrodes 914/918 may be activated together to cause larger volumes of liquid to flow into proximity with droplet dispensing electrodes. Further, the H-shaped electrode 922 and L-shaped electrodes 914/918 may be activated together with droplet dispensing electrode 926a to cause larger volumes of liquid to flow into proximity with droplet dispensing electrode 926b. Electrodes 926b and 930 may then be used to dispense a droplet. For smaller volumes, the H-shaped electrode 922 or L-shaped electrodes 914/918 may be activated individually to cause liquid to flow into proximity with electrode 926a or 926b, as the case may be. Once in proximity with the appropriate droplet dispensing electrodes 926a or 926b, droplet operations for dispensing subdroplets may be executed using droplet dispensing electrode 926a and/or 926b and droplet operations electrodes 930, e.g., by activating a row of electrodes to cause liquid to flow onto the droplet operations surface and deactivating an intermediate one or more of the electrodes to produce a subdroplet on one or more of the electrodes on the droplet operations surface.

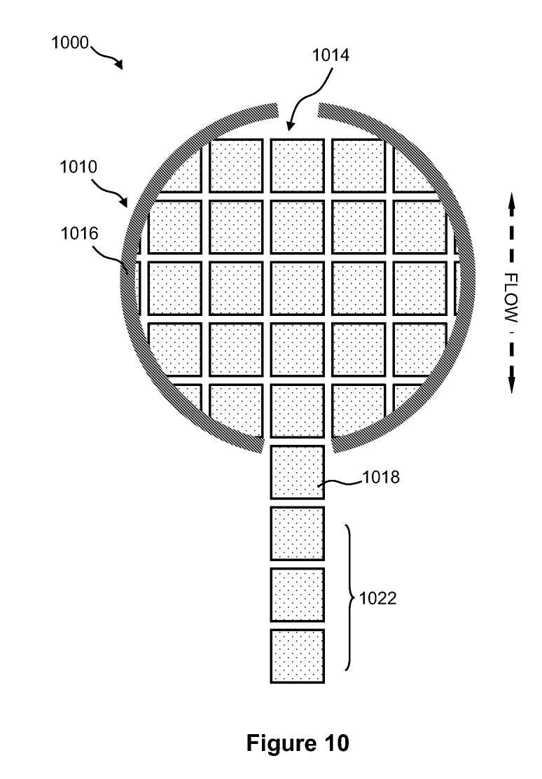

FIG. 10 illustrates a top view of yet another droplet dispensing configuration 1000 of a portion of a droplet actuator for efficiently handling varying volumes of liquid in the fluid reservoir. Droplet dispensing configuration 1000 includes a fluid reservoir 1010 that may be formed on a droplet actuator substrate or between two substrates of a droplet actuator that are separated by a gap. Disposed within fluid reservoir 1010 may be one or more electrodes for efficiently performing operations on the volume, which is variable, of liquid therein. Additionally, an opening in a barrier 1016 that serves as the boundary of fluid reservoir 1010 is adjacent to an electrode 1018 that feeds a set of transport electrodes 1022.

In one example, fluid reservoir 1010 may include electrode array 1014, which may be multiple individually-controlled electrodes that are arranged in an array, such as checkerboard pattern, within the area of fluid reservoir 1010, as shown in FIG. 10. As the volume of liquid (not shown) within fluid reservoir 1010 varies over time, due to the process of dispensing droplets via electrode 1018 and transport electrodes 1022, certain electrodes of electrode array 1014 are activated as necessary to bring the fluid into proximity with electrode 1018 so that electrodes 1018 and 1022 may be employed to dispense droplets from the fluid.

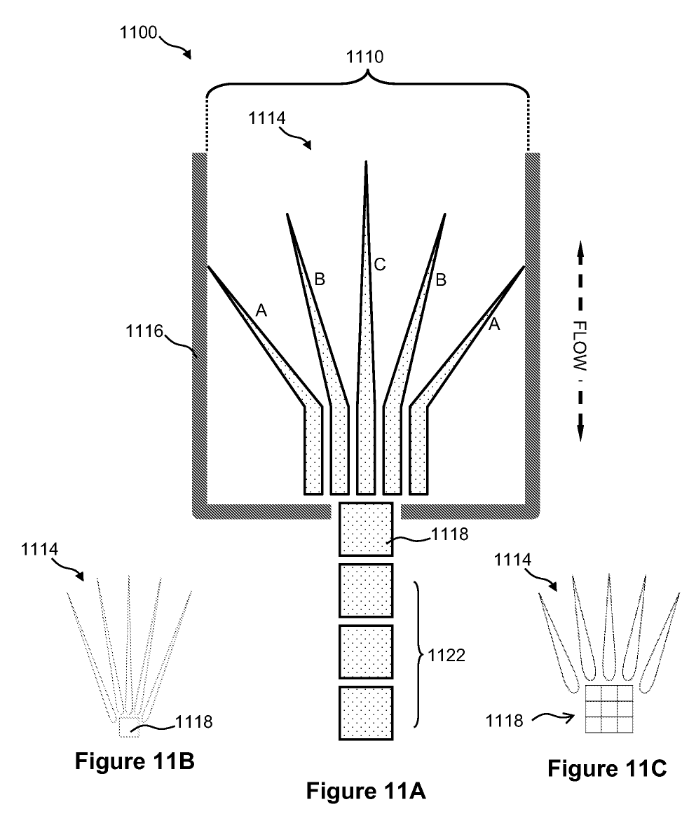

FIGS. 11A, 11B, and 11C illustrates a top view of yet another droplet dispensing configuration 1100 of a portion of a droplet actuator for efficiently handling varying volumes of liquid in the fluid reservoir. Droplet dispensing configuration 1100 includes a fluid reservoir 1110 that may be formed on a droplet actuator substrate or between two substrates of a droplet actuator that are separated by a gap. Disposed within fluid reservoir 1110 may be one or more electrodes 1114 for performing droplet dispensing operations on the various volumes of liquid therein. Additionally, an opening in a barrier 1116 that serves as the boundary of fluid reservoir 1110 is adjacent to a droplet dispensing electrode 1118 that feeds a set of transport electrodes 1122.

Electrodes 1114 may be, for example, individually-controlled elongated (e.g., finger-shaped) electrodes that are widest at the opening of fluid reservoir 1110 and narrowest opposite the opening of fluid reservoir 1110. When an electrode is activated, liquid will tend to become oriented at the widest end of the electrode in proximity with the droplet operations electrode 1118. Opposite sets of electrodes can be electrically coupled so that they can operate as single electrodes. For example, electrodes A can be electrically coupled so that they are activated and deactivated together. Similarly, electrodes A can be electrically coupled so that they are activated and deactivated together. More electrodes 1114 can be activated to handle greater volumes of fluid, and less electrodes 1114 can be activated to handle smaller volumes of fluid. As illustrated, electrodes 1114 include three electrodes, including matching pair A, matching pair B and single electrode C. Of course, any number of electrodes 114 can be used, limited only by the expediency of efficient design. In various embodiments, 2, 3, 4, 5, 6, 7, 8, 9, 10 or more electrodes 114 are provided.

In one mode of operation, electrodes 1114A, B and C are activated alone for dispensing droplets from larger volumes of liquid, electrodes 1114B and C or 1114A and B are activated alone for dispensing droplets from intermediate volumes of liquid, and electrode 1114C is activated alone for dispensing droplets from a still smaller volume of liquid.

FIG. 1B illustrates a related embodiment in which the reservoir electrodes 114 are generally elongated teardrop shapes. Having wider end proximal to the droplet operations electrode 1118 and tapering towards the tip, which is distal to the droplet operations electrode. Further, the electrodes are generally arrayed in a fan-type layout layout.

FIG. 11C illustrates another embodiment in which the droplet operations electrode 118 is divided into sub-electrodes. These sub-electrodes may be used to dispense smaller droplets from the reservoir electrodes.

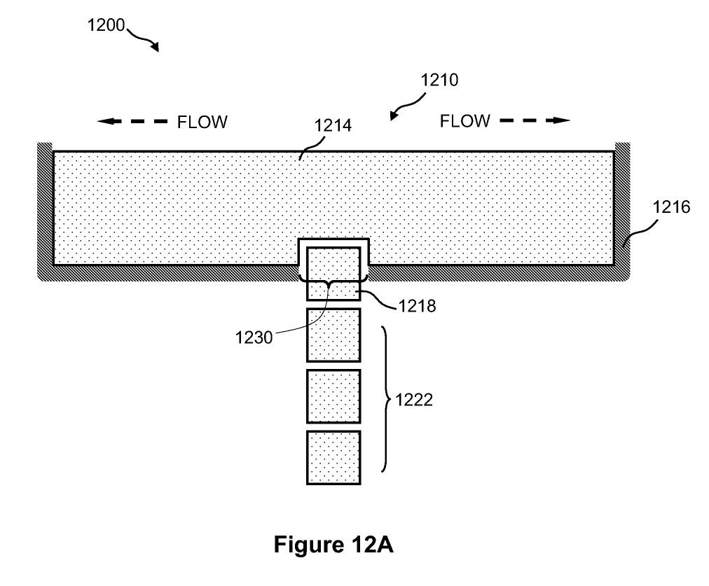

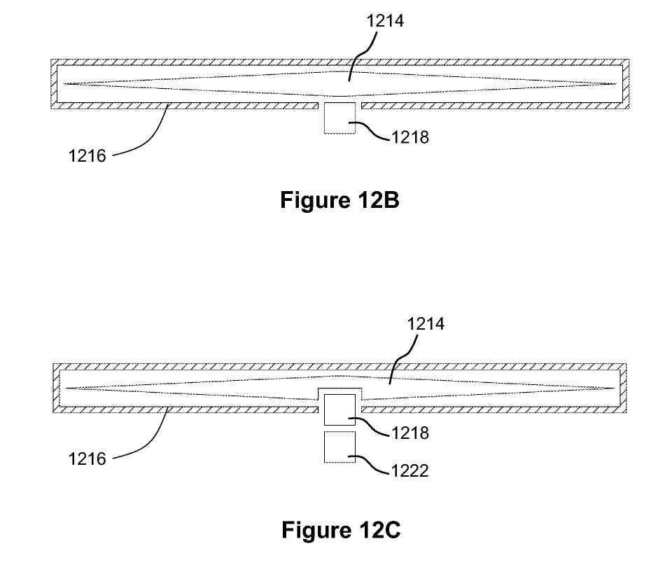

FIGS. 12A, 12B and 12C illustrates a top view of yet another droplet dispensing configuration 1200 of a portion of a droplet actuator. Droplet dispensing configuration 1200 includes a fluid reservoir 1210 that may be formed on a droplet actuator substrate or between two substrates of a droplet actuator that are separated by a gap. An electrode 1214 may be disposed within fluid reservoir 1210. An opening 1230 in a barrier 1216 serves as a fluid path from reservoir 1210 onto electrode 1218 that feeds a set of transport electrodes 1222 on a droplet operations surface.

Electrode 1214 may be, for example, an electrode that is elongated in a manner which provides pull back on the droplet during the droplet dispensing operation, where the pull back is at a right angle or acute angle to the direction in which the droplet is being dispensed. In this example, when electrode 1214 is activated during the pull-back phase of the droplet dispensing operation, the volume of liquid within fluid reservoir 1210 the liquid tends to conform to the shape of electrode 1214, resulting in a pull away from electrode 1218 and transport electrodes 1222.

FIG. 12B illustrates a similar configuration in which the reservoir electrode 1214 is thickest at a point which is proximal to electrode 1218 and tapers in a proximal direction relative to electrode 1218. FIG. 12B illustrates another similar configuration in which electrode 1218 is inset in a gap in reservoir electrode 1214.

Referring to FIG. 12C, an example of a droplet dispensing process involves activation of reservoir electrode 1214, electrode 1218 and electrode 1222, followed by deactivation of electrode 1218 to leave a droplet on electrode 1222. Similar processes are envisaged in which multiple electrodes 1222 are used to pull a longer droplet slug onto the droplet operations surface, followed by deactivation of one or more intermediate electrodes to form droplets on the droplet operations surface.

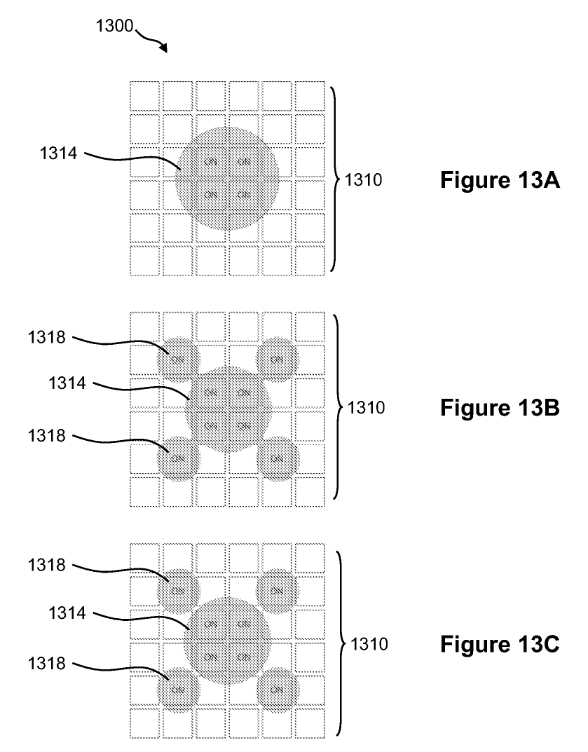

FIGS. 13A, 13B, and 13C illustrate an electrode array 1300 of a droplet actuator and illustrate a droplet dispensing process in which droplets are dispensed diagonally. For example, electrode array 1300 may be formed of an array of electrodes 1310, e.g., electrowetting electrodes. FIG. 13A shows that a droplet 1314 from which droplets are to be dispensed is held upon certain electrodes 1310 which have been activated. FIG. 13B shows that certain electrodes 1310 that are diagonal to droplet 1314 may be activated, thereby extending fingers of fluid from droplet 1314 and causing the formation of diagonally located sub-droplets 1318, as shown in FIG. 13C. The dispensing may be on a single diagonal, forming two droplets, and/or on two diagonals, forming multiple droplets. In other embodiments in which the electrode array may be formed using electrodes having more than four sides, more than four droplets may be formed.

### 7.2 Fluid Loading and Unloading Structures and Methods

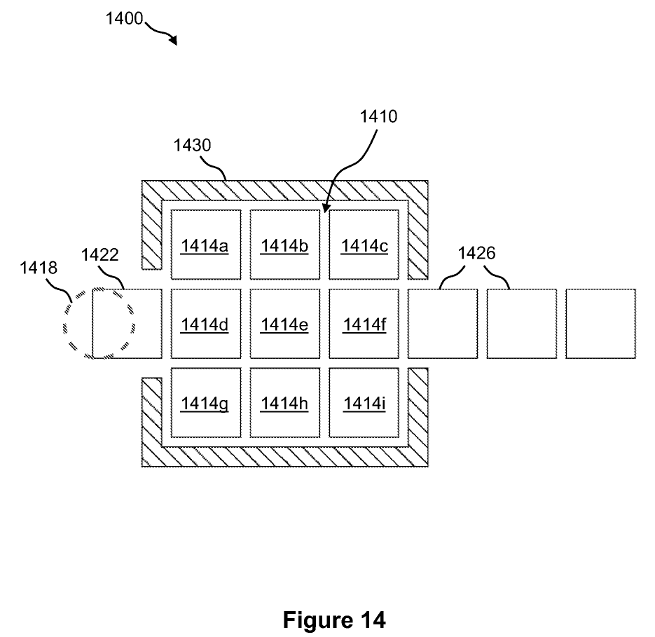

In the following embodiments of the invention, which are described in FIGS. 14 through 26C, the “opening” may, for example, be an opening in a substrate of a droplet actuator through which fluid, such as sample fluid, may be loaded into the droplet actuator and/or unloaded from the droplet actuator. Furthermore, the opening may be any shape.

FIG. 14 illustrates a top view of a reservoir droplet dispensing configuration 1400 of a droplet actuator in relation to an opening for loading/unloading fluid. Reservoir droplet dispensing configuration 1400 is associated with a fluid reservoir that may be formed between two substrates of a droplet actuator that are separated by a gap. Reservoir droplet dispensing configuration 1400 includes an electrode array 1410 that is formed of multiple electrodes. In one example, electrode array 1410 may be formed of individually controlled electrodes 1414a through 1414i that are arranged in a 3×3 array. FIG. 14 also shows an opening 1418 in a substrate of the droplet actuator. The interaction of opening 1418 with electrode array 1410 may be facilitated via a transfer electrode 1422. Transfer electrode 1422 is used to assist in the transfer of fluid that is supplied through opening 1418 onto electrode array 1410. In this example, opening 1418 is positioned to at least partially overlap with transfer electrode 1422, as shown in FIG. 14. Additionally, electrode array 1410 feeds an arrangement of electrodes 1426, e.g., electrowetting electrodes, onto which droplets (not shown) may be dispensed and by which the droplets may be subjected to droplet operations.

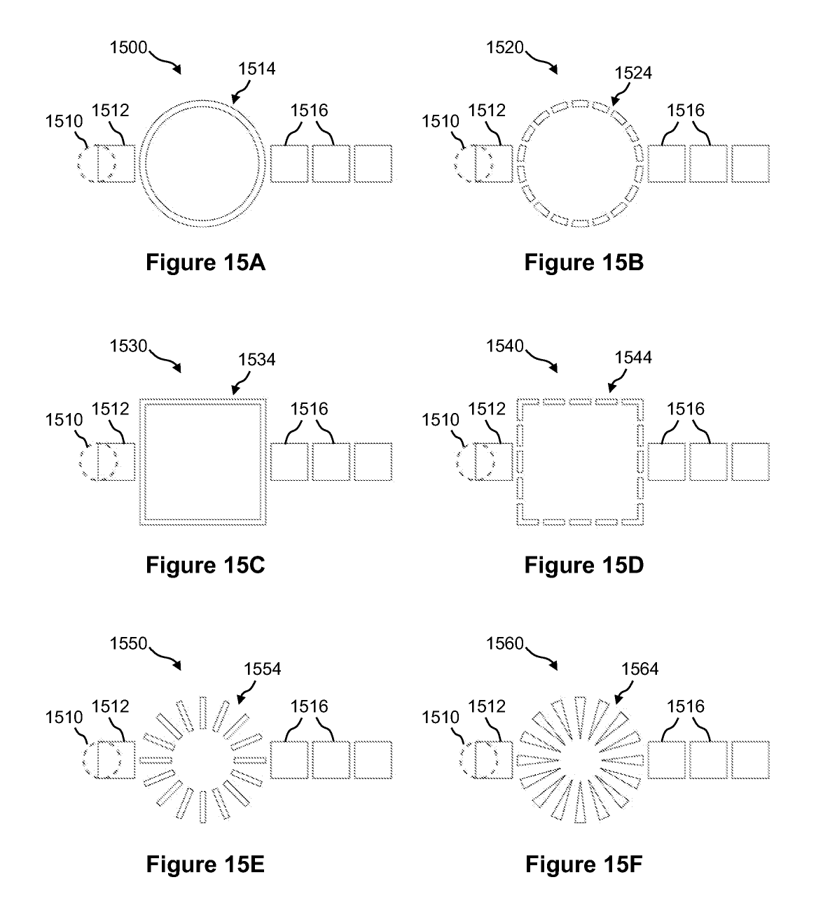

In the example reservoir droplet dispensing configuration 1400 of FIG. 14, electrode array 1410 provides a fluid reservoir that may be several times the area of a single electrode 1426. In the example shown in FIG. 14, electrode array 1410 provides a fluid reservoir that may be about 9 times the area of a single electrode 1426. Additionally, electrode array 1410 of reservoir configuration 1400 provides improved control for dispensing droplets onto electrodes 1426 via the individually controlled electrodes 1414, as compared with one large reservoir electrode. Other example reservoir configurations for providing improved control and interaction with the opening of a droplet actuator are described with reference to FIGS. 15A through 26C.

FIGS. 15A, 15B, 15C, 15D, 15E, and 15D illustrate multiple top views, respectively, of various example reservoir droplet dispensing configurations of a droplet actuator, shown in relation to an opening for loading and/or unloading fluid.

FIG. 15A shows a reservoir droplet dispensing configuration 1500 that is positioned in relation to an opening 1510. In particular, opening 1510 is positioned to at least partially overlap with a transfer electrode 1512 of reservoir configuration 1500. Transfer electrode 1512 is used to assist in the transfer of fluid that is supplied through opening 1510 onto a ring-shaped reservoir electrode 1514, e.g., circular or oval shape of any designer-defined width. Additionally, on a side of ring-shaped reservoir electrode 1514 that may be opposite to transfer electrode 1512 is an arrangement of electrodes 1516, e.g., electrowetting electrodes, onto which droplets (not shown) may be dispensed from ring-shaped reservoir electrode 1514 and subjected to droplet operations.

FIG. 15B shows a reservoir droplet dispensing configuration 1520 that is substantially the same as reservoir droplet dispensing configuration 1500 of FIG. 15A except that ring-shaped reservoir electrode 1514 of FIG. 15A is replaced with a segmented ring-shaped reservoir electrode 1524. The segment may be individually controlled or electrically coupled together to operate as a single electrode.

FIG. 15C shows a reservoir droplet dispensing configuration 1530 that is substantially the same as reservoir droplet dispensing configuration 1500 of FIG. 15A except that ring-shaped reservoir electrode 1514 of FIG. 15A is replaced with a polygon-shaped reservoir electrode 1534, e.g., square, rectangular, hexagonal, pentagonal, hexagonal, etc., shape of any designer-defined width.

FIG. 15D shows a reservoir droplet dispensing configuration 1540 that is substantially the same as reservoir droplet dispensing configuration 1500 of FIG. 15A except that ring-shaped reservoir electrode 1514 of FIG. 15A is replaced with a segmented band-shaped reservoir electrode 1544. Each segment may be individually controlled for providing further control as compared with the continuous ring-shaped reservoir electrode 1514 of FIG. 15A and/or the continuous band-shaped reservoir electrode 1534 of FIG. 15C.

FIG. 15E shows a reservoir droplet dispensing configuration 1550 that is substantially the same as reservoir droplet dispensing configuration 1500 of FIG. 15A except that ring-shaped reservoir electrode 1514 of FIG. 15A is replaced with a set of elongated electrodes 1554 that are arranged as, for example, spokes in a wheel between transfer electrode 1512 and electrodes 1514. In this example, each elongated electrode 1554 is rectangle-shaped and may be individually controlled for providing improved control.

FIG. 15F shows a reservoir droplet dispensing configuration 1560 that is substantially the same as reservoir droplet dispensing configuration 1550 of FIG. 15E except that elongated electrodes 1554 of FIG. 15E, which are rectangle-shaped, are replaced with a set of elongated electrodes 1564 that are triangle-shaped. Again, elongated electrodes 1564 are arranged as, for example, spokes in a wheel between transfer electrode 1512 and electrodes 1514, with the points of the triangles pointing inward. Each elongated electrode 1564 may be individually controlled for providing improved control.

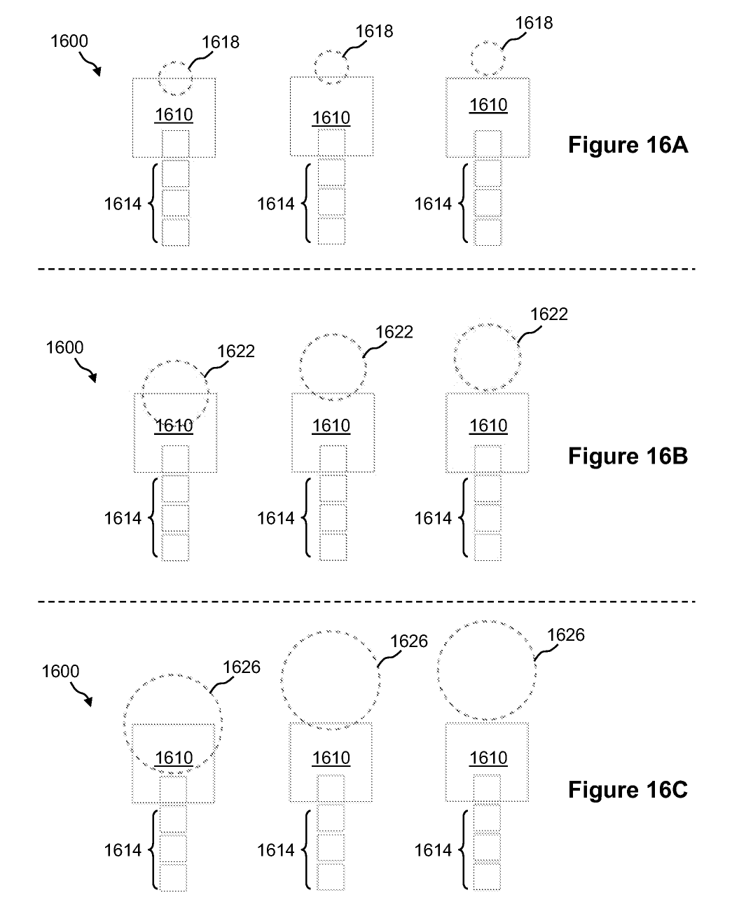

FIGS. 16A, 16B, and 16C illustrate multiple top views of certain example openings in relation to a fluid reservoir 1600 of a droplet actuator. Fluid reservoir 1600 may include a reservoir electrode 1610 feeding, for example, a line of electrodes 1614, e.g., electrowetting electrodes, onto which droplets (not shown) are dispensed from reservoir electrode 1610 and by which droplets may be subjected to droplet operations. The interaction of the reservoir electrode, such as reservoir electrode 1610, with the opening through which, for example, sample fluid may be loaded into a droplet actuator may be effected by the relative position of the opening to the reservoir electrode.

FIG. 16A shows an opening 1618 that has a diameter that may be, for example, about one third to about one half the width of reservoir electrode 1610. Additionally, FIG. 16A shows three example positions of opening 1618 relative to reservoir electrode 1610. In a first example, about half of the area of opening 1618 overlaps reservoir electrode 1610. In a second example, about less than half of the area of opening 1618 overlaps reservoir electrode 1610. In a third example, substantially none of the area of opening 1618 overlaps reservoir electrode 1610.

FIG. 16B shows an opening 1622 that has a diameter that may be, for example, about two times the diameter of opening 1618 of FIG. 16A. Additionally, FIG. 16B shows three example positions of opening 1622 relative to reservoir electrode 1610. In a first example, about half of the area of opening 1622 overlaps reservoir electrode 1610. In a second example, about less than half of the area of opening 1622 overlaps reservoir electrode 1610. In a third example, substantially none of the area of opening 1622 overlaps reservoir electrode 1610.

FIG. 16C shows an opening 1626 that has a diameter that may be, for example, about three times the diameter of opening 1618 of FIG. 16A. Additionally, FIG. 16C shows three example positions of opening 1626 relative to reservoir electrode 1610. In a first example, about half of the area of opening 1626 overlaps reservoir electrode 1610. In a second example, about less than half of the area of opening 1626 overlaps reservoir electrode 1610. In a third example, substantially none of the area of opening 1626 overlaps reservoir electrode 1610.

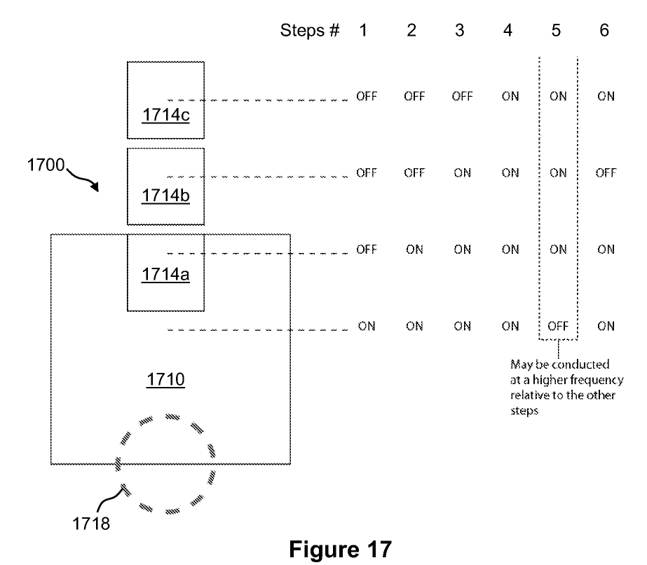

FIG. 17 illustrates a top view of a droplet dispensing configuration 1700 of a portion of a droplet actuator and illustrates a process of dispensing droplets. Droplet dispensing configuration 1700 may include a reservoir electrode 1710 that feeds, for example, a line of electrodes 1714, e.g., electrowetting electrodes 1714a, 1714b, and 1714c. Droplets (not shown) from reservoir electrode 1710 may be dispensed from reservoir electrode 1710 onto electrodes 1714 and subjected to droplet operations.

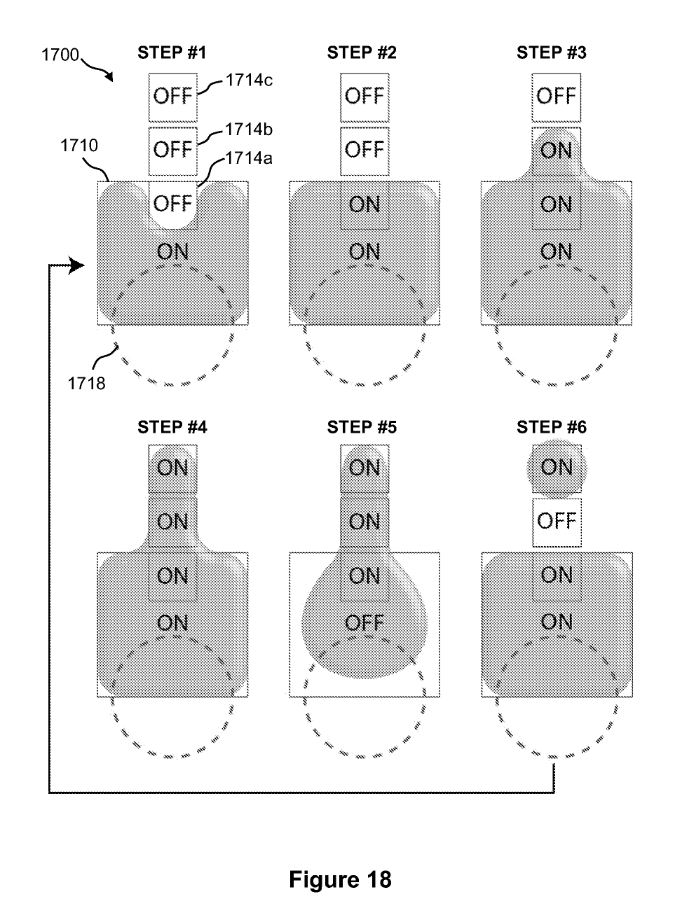

FIG. 18 illustrates another view of the droplet dispensing configuration 1700 and the process of dispensing droplets of FIG. 17.

Additionally, FIGS. 17 and 18 show electrodes 1714a, 1714b, and 1714c, where electrode 1714a is embedded within reservoir electrode 1710 and an opening 1718 near reservoir electrode 1710. Referring to FIGS. 17 and 18, the process of dispensing droplets via droplet dispensing configuration 1700 may include, but is not limited to, the following steps.

At step 1, reservoir electrode 1710=ON, electrode 1714a=OFF, electrode 1714b=OFF, and electrode 1714c=OFF. At this step, a quantity of fluid is distributed substantially across the area of reservoir electrode 1710 only and substantially no fluid and/or droplets are present atop electrodes 1714a, 1714b, and 1714c.

At step 2, reservoir electrode 1710=ON, electrode 1714a=ON, electrode 1714b=OFF, and electrode 1714c=OFF. At this step, fluid from reservoir electrode 1710 is pulled atop electrode 1714a due to the activation of electrode 1714a.

At step 3, reservoir electrode 1710=ON, electrode 1714a=ON, electrode 1714b=ON, and electrode 1714c=OFF. At this step, a finger of fluid from reservoir electrode 1710 is pulled along both electrode 1714a and electrode 1714b due to the activation of both electrode 1714a and electrode 1714b.

At step 4, reservoir electrode 1710=ON, electrode 1714a=ON, electrode 1714b=ON, and electrode 1714c=ON. At this step, the finger of fluid from reservoir electrode 1710 is pulled further along electrodes 1714 to span electrode 1714a, electrode 1714b, and electrode 1714c due to the activation of electrode 1714a, electrode 1714b, and electrode 1714c.

At step 5, reservoir electrode 1710=OFF, electrode 1714a=ON, electrode 1714b=ON, and electrode 1714c=ON. At this step, reservoir electrode 1710 is deactivated, which releases the fluid at reservoir electrode 1710 to take a shape that is suitable for dispensing a droplet. In particular, fluid atop reservoir electrode 1710 is allowed to reach equilibrium toward the slug of fluid that spans across electrode 1714a, electrode 1714b, and electrode 1714c. This step may be conducted at higher frequency relative to the other steps.

At step 6, reservoir electrode 1710=ON, electrode 1714a=ON, electrode 1714b=OFF, and electrode 1714c=ON. At this step, electrode 1714b is deactivated and reservoir electrode 1710 is reactivated, which pulls a portion of the slug back toward reservoir electrode 1710 and causes the slug of liquid to split at electrode 1714b, which is serving as the electrode, leaving behind a droplet at electrode 1714c.

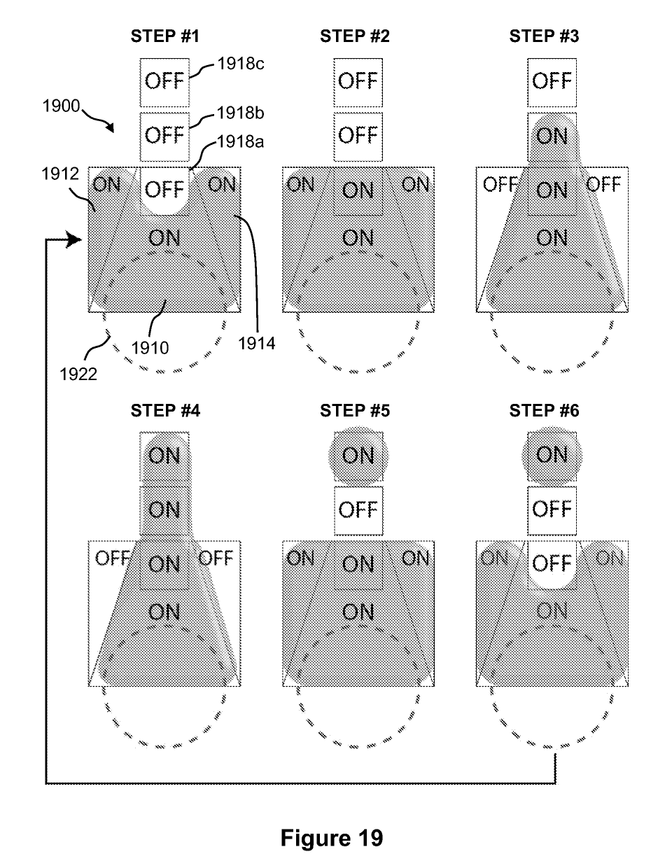

FIG. 19 illustrates a top view of another droplet dispensing configuration 1900 of a portion of a droplet actuator and illustrates another process of dispensing droplets. Droplet dispensing configuration 1900 may include a central reservoir electrode 1910, a first side reservoir electrode 1912, and a second side reservoir electrode 1914. Central reservoir electrode 1910 may have a tapered geometry, as shown in FIG. 19. First side reservoir electrode 1912 and second side reservoir electrode 1914 may be triangular in shape and fitted to central reservoir electrode 1910, as shown in FIG. 19. The combination of central reservoir electrode 1910, first side reservoir electrode 1912, and second side reservoir electrode 1914 forms a substantially rectangular or square reservoir electrode that is segmented for improved control. In particular, the segments are shaped in a manner to assist in the droplet dispensing process.

The narrow end of central reservoir electrode 1910 feeds, for example, a line of electrodes 1918, e.g., electrowetting electrodes 1918a, 1918b, and 1918c, onto which droplets are dispensed from central reservoir electrode 1910 and by which droplets may be subjected to droplet operations. More specifically, FIG. 19 shows electrodes 1918a, 1918b, and 1918c, where electrode 1918a is embedded within the narrow end of central reservoir electrode 1910 and an opening 1922 near central reservoir electrode 1910. Referring to FIG. 19, the process of dispensing droplets via droplet dispensing configuration 1900 may include, but is not limited to, the following steps.

At step 1, central reservoir electrode 1910=ON, first side reservoir electrode 1912=ON, second side reservoir electrode 1914=ON, electrode 1918a=OFF, electrode 1918b=OFF, and electrode 1918c=OFF. At this step, a quantity of fluid is distributed substantially across the combined area of central reservoir electrode 1910, first side reservoir electrode 1912, and second side reservoir electrode 1914 and substantially no fluid and/or droplets are present atop electrodes 1918a, 1918b, and 1918c.

At step 2, central reservoir electrode 1910=ON, first side reservoir electrode 1912=ON, second side reservoir electrode 1914=ON, electrode 1918a=ON, electrode 1918b=OFF, and electrode 1918c=OFF. At this step, fluid from central reservoir electrode 1910 is pulled atop electrode 1918a due to the activation of electrode 1918a.

At step 3, central reservoir electrode 1910=ON, first side reservoir electrode 1912=OFF, second side reservoir electrode 1914=OFF, electrode 1918a=ON, electrode 1918b=ON, and electrode 1918c=OFF. At this step, a finger of fluid from central reservoir electrode 1910 is pulled along both electrode 1918a and electrode 1918b due to the activation of both electrode 1918a and electrode 1918b. Additionally, because first side reservoir electrode 1912 and second side reservoir electrode 1914 are deactivated, the fluid at central reservoir electrode 1910 takes on a shape that is suitable to assist in the droplet dispensing process, as shown in FIG. 19.

At step 4, central reservoir electrode 1910=ON, first side reservoir electrode 1912=OFF, second side reservoir electrode 1914=OFF, electrode 1918a=ON, electrode 1918b=ON, and electrode 1918c=ON. At this step, the finger of fluid from central reservoir electrode 1910 is pulled further along electrodes 1918 to span electrode 1918a, electrode 1918b, and electrode 1714c due to the activation of electrode 1918a, electrode 1918b, and electrode 1918c and the deactivation of first side reservoir electrode 1912 and second side reservoir electrode 1914.

At step 5, central reservoir electrode 1910=ON, first side reservoir electrode 1912=ON, second side reservoir electrode 1914=ON, electrode 1918a=ON, electrode 1918b=OFF, and electrode 1918c=ON. At this step, electrode 1918b is deactivated and the pull of central reservoir electrode 1910, which is now activated, draws a portion of the slug back toward central reservoir electrode 1910 and causes the slug of liquid to split at electrode 1918b, which is serving as the electrode, leaving a droplet at electrode 1918c.

At step 6, central reservoir electrode 1910=ON, first side reservoir electrode 1912=ON, second side reservoir electrode 1914=ON, electrode 1918a=OFF, electrode 1918b=OFF, and electrode 1918c=ON. At this step, the volume of fluid is pulled back across the combined area of central reservoir electrode 1910, first side reservoir electrode 1912, and second side reservoir electrode 1914 and no fluid is present atop electrodes 1918a and 1918b. A droplet remains at electrode 1918c.

Referring to steps 1 through 6 of the process of dispensing droplets via droplet dispensing configuration 1900, the necessity to entirely deactivate the reservoir electrode is avoided. More specifically, central reservoir electrode 1910 remains activated throughout all steps of electrode activation sequence 1900 and first side reservoir electrode 1912 and second side reservoir electrode 1914 only are sequenced on and off.

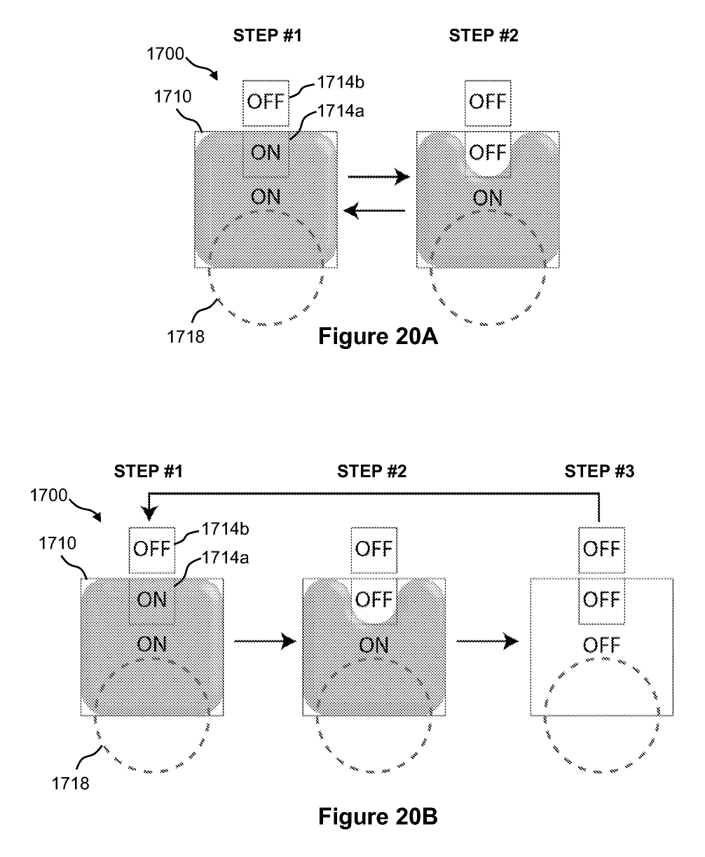

FIG. 20A illustrates another top view of droplet dispensing configuration 1700 of FIG. 17 and illustrates a process of agitating droplets and/or priming the fluid reservoir in a droplet actuator. Referring to FIG. 20A, the process of agitating droplets via droplet dispensing configuration 1700 may include, but is not limited to, the following steps.

At step 1, reservoir electrode 1710=ON, electrode 1714a=ON, and electrode 1714b=OFF. In this step, a quantity of fluid is distributed substantially across the combined area of reservoir electrode 1710 and electrodes 1714a and no fluid is present atop 1714b.

At step 2, reservoir electrode 1710=ON, electrode 1714a=OFF, and electrode 1714b=OFF. In this step, electrode 1714a is deactivated which causes fluid at electrode 1714a to be drawn back to reservoir electrode 1714a and substantially no fluid is present atop 1714b.

The process of agitating droplets via droplet dispensing configuration 1700 alternates between steps 1 and 2 in order to achieve a droplet agitation operation. Alternatively, alternating between steps 1 and 2 may be used in order to prime the liquid that is supplied via opening 1718 onto reservoir electrode 1710. This priming operation may be carried out at the same time that other droplet operations are being performed.

FIG. 20B illustrates yet another top view of droplet dispensing configuration 1700 of FIG. 17 and illustrates a process of agitating fluid in a droplet actuator. The process of agitating fluid via droplet dispensing configuration 1700 may include, but is not limited to, the following steps.

At step 1, reservoir electrode 1710=ON, electrode 1714a=ON, and electrode 1714b=OFF. In this step, a quantity of fluid is distributed substantially across the combined area of reservoir electrode 1710 and electrodes 1714a and substantially no fluid is present atop electrode 1714b.

At step 2, reservoir electrode 1710=ON, electrode 1714a=OFF, and electrode 1714b=OFF. In this step, electrode 1714a is deactivated which causes fluid at electrode 1714a to be drawn back to reservoir electrode 1714a and substantially no fluid is present atop electrode 1714b.

At step 3, reservoir electrode 1710=OFF, electrode 1714a=OFF, and electrode 1714b=OFF. In this step, by deactivating reservoir electrode 1710, the fluid thereon is allowed to be substantially evacuated through opening 1718, which provides a mechanism for disaggregating beads (not shown) in a fluid reservoir.

The process of agitating fluid via droplet dispensing configuration 1700 may repeatedly loop through steps 1, 2, and 3 in order to achieve a droplet agitation operation. For example, once beads (not shown) are loaded into the fluid reservoir, such as reservoir electrode 1710, the beads tend to settle onto the surface of the fluid reservoir due to gravity. However, in order to resuspend them for use in an assay, the beads can be resuspended by loading fluid into the droplet actuator via opening 1718 and then returning the fluid back through opening 1718 (e.g., by switching off reservoir electrode 1710 in step 3). This action causes recirculation and resuspends the beads.

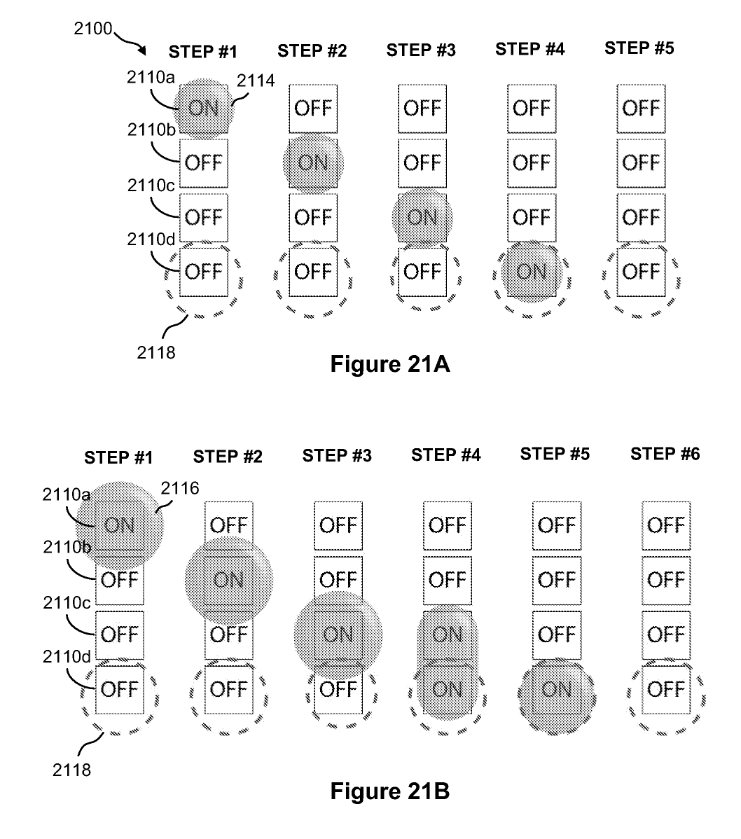

FIG. 21A illustrates a top view of a droplet dispensing configuration 2100 of a portion of a droplet actuator and illustrates a process of disposing of a 1× size droplet in a droplet actuator. Droplet dispensing configuration 2100 includes a line of electrodes 2110 (e.g., electrowetting electrodes 2110a, 2110b, 2110c, and 2110d for disposing of a 1× size droplet 2114 through an opening 2118 of a droplet actuator. In this example, opening 2118 is located in close proximity to electrode 2110d. The 1× size refers to the approximate footprint of the droplet in relation to the approximate area of a single electrode 2110. The process of disposing of a 1× size droplet via droplet dispensing configuration 2100 may include, but is not limited to, the following steps.

At step 1, electrode 2110a=ON, electrode 2110b=OFF, electrode 2110c=OFF, and electrode 2110d=OFF. In this step, 1× size droplet 2114 is held at electrode 2110a due to the activation of electrode 2110a only.

At step 2, electrode 2110a=OFF, electrode 2110b=ON, electrode 2110c=OFF, and electrode 2110d=OFF. In this step, electrode 2110a is deactivated and its neighbor, electrode 2110b, is activated. This causes 1× size droplet 2114 to move from electrode 2110a to electrode 2110b, which is in a direction that is toward opening 2118.

At step 3, electrode 2110a=OFF, electrode 2110b=OFF, electrode 2110c=ON, and electrode 2110d=OFF. In this step, electrode 2110b is deactivated and its neighbor, electrode 2110c, is activated. This causes 1× size droplet 2114 to move from electrode 2110b to electrode 2110c, which is in a direction that is toward opening 2118.

At step 4, electrode 2110a=OFF, electrode 2110b=OFF, electrode 2110c=OFF, and electrode 2110d=ON. In this step, electrode 2110c is deactivated and its neighbor, electrode 2110d, is activated. This causes 1× size droplet 2114 to move from electrode 2110c to electrode 2110d, which is located in close proximity to opening 2118.

At step 5, electrode 2110a=OFF, electrode 2110b=OFF, electrode 2110c=OFF, and electrode 2110d=OFF. In this step, electrode 2110d is deactivated, which allows 1× size droplet 2114 to be evacuated from the droplet actuator (i.e., disposed of) through opening 2118.

FIG. 21B illustrates another top view of the droplet dispensing configuration 2100 of FIG. 21A and illustrates a process of disposing of a 2× size droplet in a droplet actuator. For example, FIG. 21B shows a 2× size droplet 2116 atop droplet dispensing configuration 2100. The 2× size refers to the approximate footprint of the droplet in relation to the approximate area of a single electrode 2110. The process of disposing of a 2× size droplet via droplet dispensing configuration 2100 may include, but is not limited to, the following steps.

At step 1, electrode 2110a=ON, electrode 2110b=OFF, electrode 2110c=OFF, and electrode 2110d=OFF. In this step, 2× size droplet 2116 is held at electrode 2110a due to the activation of electrode 2110a only.

At step 2, electrode 2110a=OFF, electrode 2110b=ON, electrode 2110c=OFF, and electrode 2110d=OFF. In this step, electrode 2110a is deactivated and its neighbor, electrode 2110b, is activated. This causes 2× size droplet 2116 to move from electrode 2110a to electrode 2110b, which is in a direction that is toward opening 2118.

At step 3, electrode 2110a=OFF, electrode 2110b=OFF, electrode 2110c=ON, and electrode 2110d=OFF. In this step, electrode 2110b is deactivated and its neighbor, electrode 2110c, is activated. This causes 2× size droplet 2116 to move from electrode 2110b to electrode 2110c, which is in a direction that is toward opening 2118.

At step 4, electrode 2110a=OFF, electrode 2110b=OFF, electrode 2110c=ON, and electrode 2110d=ON. In this step, both electrode 2110c and its neighbor, electrode 2110d, are activated. This causes 2× size droplet 2116 to change shape and spread across both electrode 2110c and electrode 2110d, which creates a slug of fluid that is located in close proximity to opening 2118.

At step 5, electrode 2110a=OFF, electrode 2110b=OFF, electrode 2110c=OFF, and electrode 2110d=ON. In this step, electrode 2110c is deactivated, which leaves electrode 2110d only activated. This releases a portion of the volume of 2× size droplet 2116 to be evacuated from the droplet actuator (i.e., disposed of) through opening 2118, which leaves the balance of the volume of 2× size droplet 2116 at electrode 2110d.

At step 6, electrode 2110a=OFF, electrode 2110b=OFF, electrode 2110c=OFF, and electrode 2110d=OFF. In this step, electrode 2110d is deactivated, which allows the balance of the volume of 2× size droplet 2116 from step 5 to be evacuated from the droplet actuator (i.e., disposed of) through opening 2118.

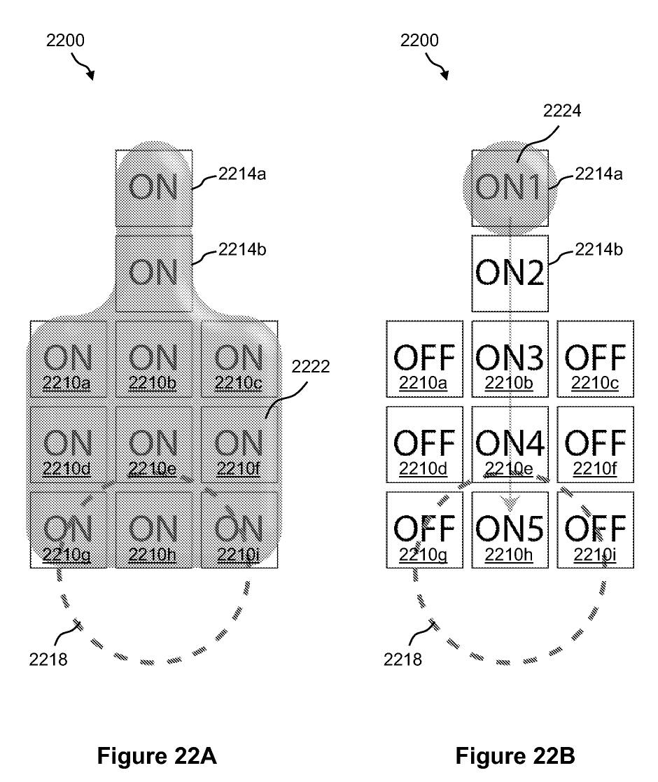

FIG. 22A illustrates a top view of a dual-purpose droplet dispensing configuration 2200 of a portion of a droplet actuator and illustrates a process of dispensing droplets in a droplet actuator. Dual-purpose droplet dispensing configuration 2200 includes an array of multiple electrodes 2210 that serve as the fluid reservoir of a droplet actuator (not shown). In one example, electrodes 2210a through 2210i are arranged in a 3×3 array, as shown in FIG. 22A. Arranged on one side of the array of electrodes 2210 may be a line of electrodes 2214, such as electrodes 2214a and 2214b, which may be, for example, electrowetting electrodes. Electrodes 2210 and electrodes 2214 may be individually controlled. Located, for example, near the side of the array of electrodes 2210 that is opposite electrodes 2214 may be an opening 2218. Additionally, FIG. 22A shows all electrodes 2210 and electrodes 2214 in an activated state and a quantity of fluid 2222 that is distributed atop the combined area of electrodes 2210 and electrodes 2214.

FIG. 22A shows dual-purpose droplet dispensing configuration 2200 in one step of a droplet dispensing operation in a droplet actuator. In one example, the droplet dispensing process may be substantially the same as the droplet dispensing process that is described with reference to FIGS. 17 and 18.

FIG. 22B illustrates another top view of dual-purpose droplet dispensing configuration 2200 of FIG. 22A and illustrates a process of disposing of droplets in a droplet actuator.

FIG. 22B shows a droplet 2224 that is located atop electrode 2214a. In this example, droplet 2224 is to be transported from electrode 2214a to electrode 2214a, then to electrode 2210b, then to electrode 2210e, then to electrode 2210h, and evacuated from the droplet actuator (i.e., disposed of) through opening 2218. The droplet disposal process may be substantially the same as the droplet disposal process that is described with reference to FIG. 21A.

An aspect of the dual-purpose droplet dispensing configuration 2200 of FIGS. 22A and 22B is that the same droplet dispensing configuration may be suited for both a droplet dispensing operation and a droplet disposal operation.

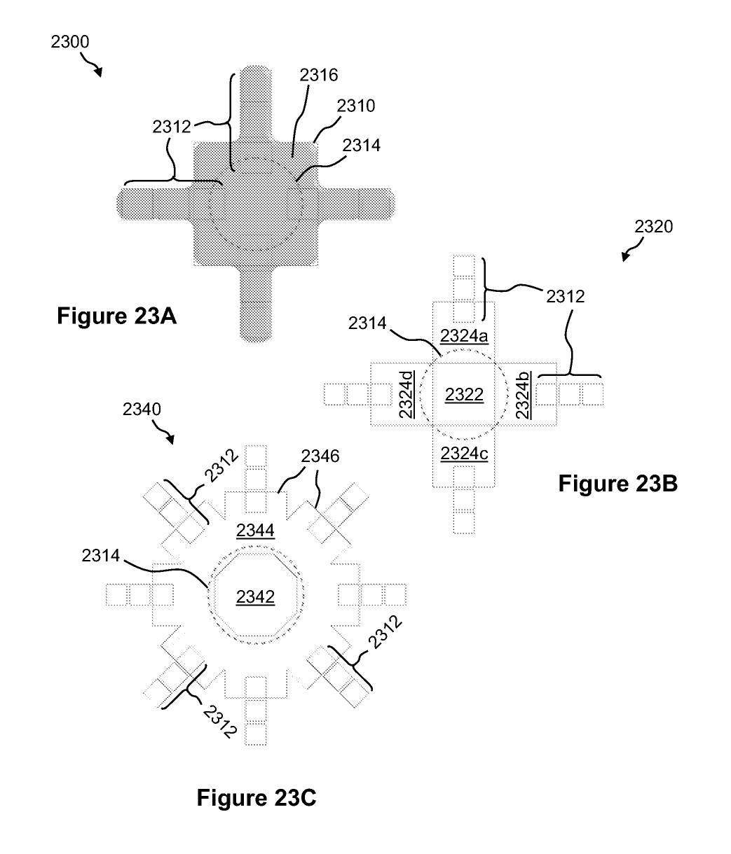

FIG. 23A illustrates a top view of an example droplet dispensing configuration 2300 for dispensing droplets in multiple directions from a single reservoir in a droplet actuator. Droplet dispensing configuration 2300 may include a central reservoir electrode 2310, which may be, for example, square or rectangular in shape, and multiple lines of electrodes 2312. For example, a first line of electrodes 2312 may be arranged at a first side of central reservoir electrode 2310, a second line of electrodes 2312 may be arranged at a second side of central reservoir electrode 2310, a third line of electrodes 2312 may be arranged at a third side of central reservoir electrode 2310, and a fourth line of electrodes 2312 may be arranged at a fourth side of central reservoir electrode 2310, as shown in FIG. 23A. In this example, the first electrode 2312 of each line of electrodes 2312 may be embedded in central reservoir electrode 2310.

Additionally, an opening 2314 is substantially centrally located in relation to central reservoir electrode 2310. The diameter of opening 2314 may be suitably sized such that a portion of opening 2314 may overlap the first electrode 2312 of each line of electrodes 2312. In this way, the presence or absence of central reservoir electrode 2310 may be optional.

An aspect of droplet dispensing configuration 2300 of FIG. 23A is that it provides a single reservoir from which droplets may be dispensed in multiple directions, such as, but not limited to, four directions. Another aspect of droplet dispensing configuration 2300 is that the presence or absence of the central electrode, such as central reservoir electrode 2310, may be optional.

FIG. 23B illustrates a top view of another example droplet dispensing configuration 2320 for dispensing droplets in multiple directions from a single reservoir in a droplet actuator. Droplet dispensing configuration 2320 may include a central reservoir electrode 2322, which may be, for example, square or rectangular in shape, and multiple side electrodes 2324 for feeding multiple lines of electrodes 2312, which are described in FIG. 23A. For example, a side electrode 2324a that feeds a first line of electrodes 2312 may be arranged at a first side of central reservoir electrode 2322, a side electrode 2324b that feeds a second line of electrodes 2312 may be arranged at a second side of central reservoir electrode 2322, a side electrode 2324c that feeds a third line of electrodes 2312 may be arranged at a third side of central reservoir electrode 2322, a side electrode 2324d that feeds a fourth line of electrodes 2312 may be arranged at a fourth side of central reservoir electrode 2322, as shown in FIG. 23B. In this example, the first electrode 2312 of each line of electrodes 2312 may be embedded in each of the respective side electrodes 2324.

Additionally, opening 2314 is substantially centrally located in relation to central reservoir electrode 2322. The diameter of opening 2314 may be suitably sized such that a portion of opening 2314 may overlap each of the side electrodes 2324. In this way, the presence or absence of central reservoir electrode 2322 may be optional.

An aspect of droplet dispensing configuration 2320 of FIG. 23B is that it provides a single reservoir from which droplets may be dispensed in multiple directions, such as, but not limited to, four directions. Another aspect of droplet dispensing configuration 2320 is that the presence or absence of the central electrode, such as central reservoir electrode 2322, may be optional.

FIG. 23C illustrates a top view of yet another example droplet dispensing configuration 2340 for dispensing droplets in multiple directions from a single reservoir in a droplet actuator. Droplet dispensing configuration 2340 may include a central reservoir electrode 2342, which may be, for example, square, rectangular, circular, hexagonal, or octagonal in shape, and a distribution electrode 2344 that substantially surrounds central reservoir electrode 2342. Furthermore, the geometry of distribution electrode 2344 has multiple platforms 2346 (see FIG. 23C) for feeding multiple lines of electrodes 2312, which are described in FIG. 23A.

For example, a first platform 2346 of distribution electrode 2344 feeds a first line of electrodes 2312, a second platform 2346 of distribution electrode 2344 feeds a second line of electrodes 2312, a third platform 2346 of distribution electrode 2344 feeds a third line of electrodes 2312, a fourth platform 2346 of distribution electrode 2344 feeds a fourth line of electrodes 2312, a fifth platform 2346 of distribution electrode 2344 feeds a fifth line of electrodes 2312, a sixth platform 2346 of distribution electrode 2344 feeds a sixth line of electrodes 2312, a seventh platform 2346 of distribution electrode 2344 feeds a seventh line of electrodes 2312, an eighth platform 2346 of distribution electrode 2344 feeds an eighth line of electrodes 2312, as shown in FIG. 23C. In this example, the first electrode 2312 of each line of electrodes 2312 may be embedded in each of the respective platforms 2346.

Additionally, opening 2314 is substantially centrally located in relation to central reservoir electrode 2342. The diameter of opening 2314 may be suitably sized such that a portion of opening 2314 may overlap a portion of distribution electrode 2344. In this way, the presence or absence of central reservoir electrode 2342 may be optional.

An aspect of droplet dispensing configuration 2340 of FIG. 23C is that it provides a single reservoir from which droplets may be dispensed in multiple directions, such as, but not limited to, eight directions. Another aspect of droplet dispensing configuration 2340 is that the presence or absence of the central electrode, such as central reservoir electrode 2342, may be optional.

Referring to FIGS. 23A, 23B, and 23C, the geometries of the reservoir configurations are not limited to those shown in FIGS. 23A, 23B, and 23C only. In other embodiments, the geometries of the reservoir configurations may be modified to any shape that is suitable for dispensing droplets in any number of directions. Additionally, opening 2314 is not limited to circular. Alternatively, opening 2314 may be any geometry that is suited to correspond with the geometries of the reservoir configurations.

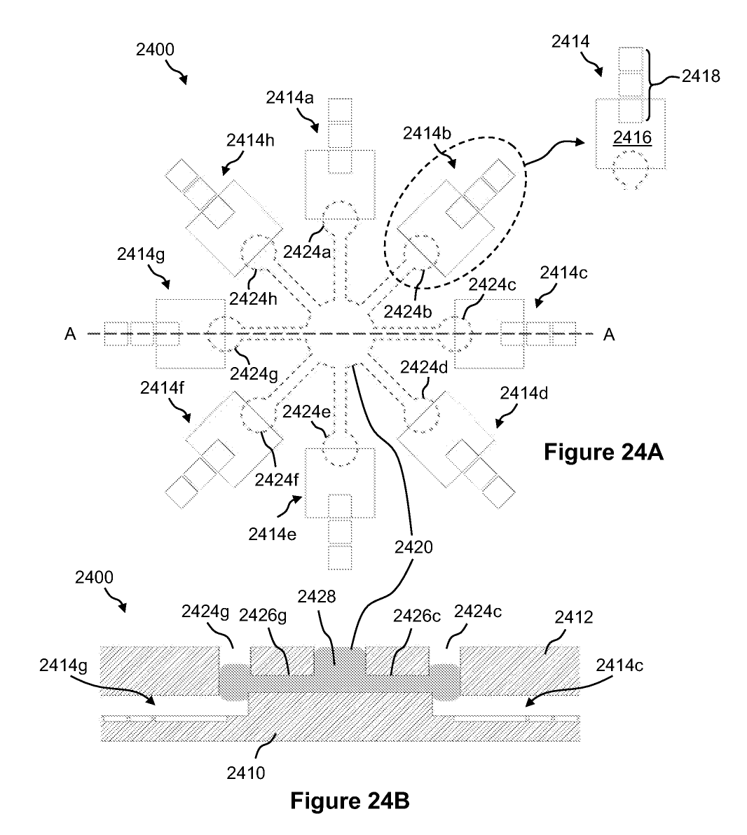

FIG. 24A illustrates a top view of a portion of a droplet actuator 2400 for parallel distribution of fluid to multiple fluid reservoirs using a single opening. Additionally, FIG. 24B illustrates a cross-sectional view of droplet actuator 2400 taken along line AA of FIG. 24A. Referring to FIGS. 24A and 24B, droplet actuator 2400 may include a bottom substrate 2410 that is separated from a top substrate 2412 by a gap. A set of multiple droplet dispensing configurations 2414 may be associated with bottom substrate 2410. In one example, droplet actuator 2400 may include droplet dispensing configurations 2414a through 2414h, as shown in FIG. 24A. Furthermore, each droplet dispensing configuration 2414 may be formed of a reservoir electrode 2416 that feeds a line of electrodes 2418, e.g., electrowetting electrodes.

Droplet actuator 2400 further includes a central opening 2420 that is fluidly connected to multiple openings 2424, which correspond to the respective droplet dispensing configurations 2414, via respective fluid channels 2426. For example, central opening 2420 is fluidly connected to openings 2424a through 2424h via fluid channels 2426a through 2426h, respectively. Additionally, openings 2424a through 2424h correspond to droplet dispensing configurations 2414a through 2414h, respectively. Furthermore, at least a portion of openings 2424a through 2424h may overlap each respective reservoir electrode 2416 of droplet dispensing configurations 2414a through 2414h, as shown in FIGS. 24A and 24B.

In operation, a quantity of fluid, such as a quantity of sample fluid 2428, may be loaded into droplet actuator 2400 via central opening 2420. Fluid 2428 then flows in a substantially simultaneous manner through fluid channels 2426 and fills openings 2424a through 2424h, thereby supplying fluid 2428 in a substantially simultaneous manner to each respective reservoir electrode 2416 of the corresponding droplet dispensing configurations 2414a through 2414h.

Optionally, a quantity of fluid 2428 may be loaded into droplet actuator 2400 via any one of the openings 2424a through 2424h. However, in this instance, droplet dispensing configurations 2414a through 2414h may not be supplied with fluid 2428 in a substantially simultaneous manner, as fluid 2428 may reach the respective droplet dispensing configurations 2414 at slightly different times. Optionally, a quantity of fluid 2428 may be loaded into a certain droplet dispensing configuration 2414 only via its associated opening 2424. For example, droplet dispensing configuration 2414c only may be loaded via opening 2424c.

In another embodiment, openings 2424 are absent from droplet actuator 2400. Instead, fluid may be supplied from central opening 2420 only, then flow through fluid channels 2426 to droplet dispensing configurations 2414.

In yet another embodiment, the fluid paths, such as fluid channels 2426, may lead to any type of electrode, as the invention is not limited to the fluid paths leading to reservoir electrodes only.

FIG. 25A illustrates a top view of a portion of a droplet actuator 2500 for serial distribution of fluid to multiple fluid reservoirs using a single opening. Additionally, FIG. 25B illustrates a cross-sectional view of droplet actuator 2500 taken along line BB of FIG. 25A.

Referring to FIGS. 25A and 25B, droplet actuator 2500 may include a bottom substrate 2510 that is separated from a top substrate 2512 by a gap. A set of multiple droplet dispensing configurations 2514 may be associated with bottom substrate 2510. In one example, droplet actuator 2500 may include droplet dispensing configurations 2514a through 2514c, as shown in FIG. 25A. Furthermore, each droplet dispensing configuration 2514 may be formed of a reservoir electrode 2516 that feeds a line of electrodes 2518, e.g., electrowetting electrodes.

Droplet actuator 2500 further includes a fluid channel 2520 that is fluidly connected to multiple openings 2522, which correspond respectively to the multiple droplet dispensing configurations 2514. For example, fluid channel 2520 is fluidly connected to openings 2522a through 2522c, which correspond to droplet dispensing configurations 2514a through 2514c, respectively. Furthermore, at least a portion of openings 2522a through 2522c may overlap each respective reservoir electrode 2516 of droplet dispensing configurations 2514a through 2514c, as shown in FIGS. 25A and 25B.

In operation, a quantity of fluid, such as a quantity of sample fluid 2528, may be loaded into droplet actuator 2400 via fluid channel 2520. Fluid 2428 then flows through fluid channel 2520 and reaches openings 2522a through 2522c in a substantially serial manner, thereby supplying fluid 2528 in a substantially sequential manner to each respective reservoir electrode 2516 of the corresponding droplet dispensing configurations 2514a through 2514c. In one example, via fluid channel 2520, fluid 2428 may first reach droplet dispensing configuration 2514a, then droplet dispensing configuration 2514b, and then droplet dispensing configuration 2514c.

In another embodiment, the fluid path, such as fluid channel 2520, may lead to any type of electrode, as the invention is not limited to the fluid path leading to reservoir electrodes only.

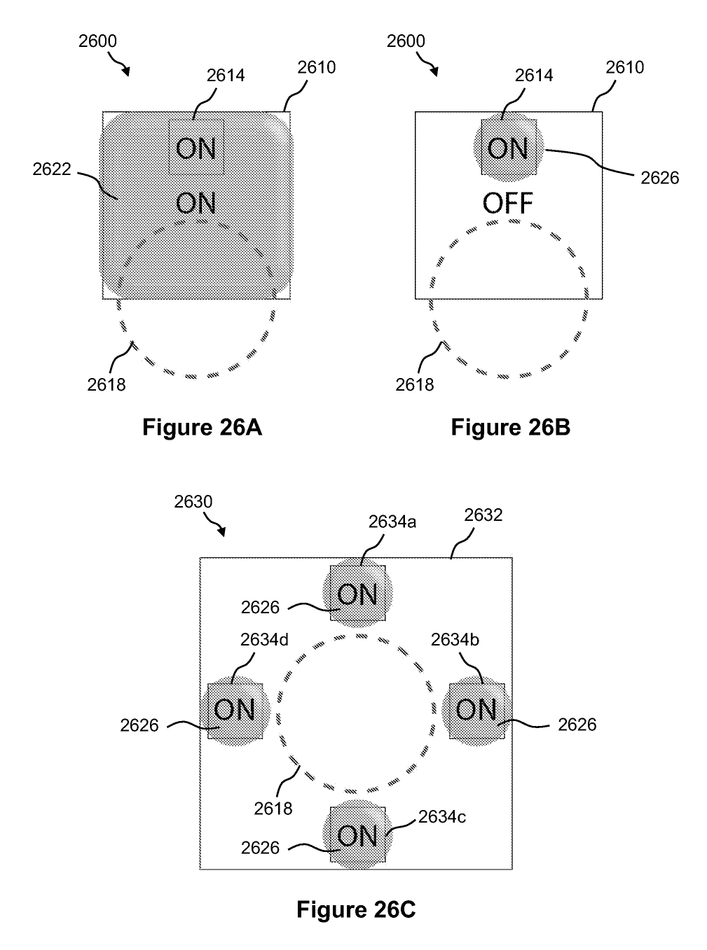

FIGS. 26A and 26B illustrate top views of an example droplet dispensing configuration 2600 of a droplet actuator that includes a droplet forming electrode that is embedded in a larger reservoir electrode. Droplet dispensing configuration 2600 may include a reservoir electrode 2610 having a droplet forming electrode 2614 embedded therein, as shown in FIGS. 26A and 26B. Reservoir electrode 2610 may be, for example, several times larger in area than droplet forming electrode 2614. Additionally, FIGS. 26A and 26B show an opening 2618 that is associated with reservoir electrode 2610.

In FIG. 26A, both reservoir electrode 2610 and droplet forming electrode 2614 are activated. Consequently, a quantity of fluid, such as sample fluid 2622, that is supplied via opening 2618 is atop the combined area of reservoir electrode 2610 and droplet forming electrode 2614.

In FIG. 26B, reservoir electrode 2610 is deactivated and droplet forming electrode 2614 only is activated. Consequently, the quantity of fluid 2622 that is atop reservoir electrode 2610 (see FIG. 26A) may be evacuated through opening 2618, leaving a droplet 2626 atop droplet forming electrode 2614 only.

FIG. 26C illustrates a top view of an example droplet dispensing configuration 2630 of a droplet actuator that includes multiple droplet forming electrodes that are embedded in a larger reservoir electrode. Droplet dispensing configuration 2630 may include a reservoir electrode 2632 having multiple droplet forming electrodes 2634 (e.g., droplet forming electrodes 2634a, 2634b, 2634c, and 2634d) embedded therein, as shown in FIG. 26C. Reservoir electrode 2632 may be, for example, several times larger in area than each droplet forming electrode 2634. Additionally, FIG. 26C shows opening 2618 that is positioned substantially in a central area of reservoir electrode 2632.

In FIG. 26C, reservoir electrode 2632 is deactivated and droplet forming electrodes 2634a, 2634b, 2634c, and 2634d are activated. Consequently, any quantity of fluid that may have been atop reservoir electrode 2632 may be evacuated through opening 2618, leaving a droplet 2626 atop droplet forming electrodes 2634a, 2634b, 2634c, and 2634d only.

The invention is not limited to the example embodiments shown in FIGS. 1 through 26A, 26B, and 26C. The scope of the invention may include any combinations of the example embodiments shown in FIGS. 1 through 26A, 26B, and 26C. Additionally, variations of the example embodiments shown in FIGS. 1 through 26A, 26B, and 26C may utilize, for example, pressure, electrowetting, gravity effect, capillary force, and any combinations thereof as the energy source for moving a volume of liquid in a droplet actuator. Furthermore, variations of the example embodiments shown in FIGS. 1 through 26A, 26B, and 26C may include fluid reservoirs, electrodes, and openings of any size, shape, and/or geometry, such as but not limited to, rectangular, square, circular, oval, hexagonal, and octagonal.

### 7.3 Droplet Actuator

For examples of droplet actuator architectures that are suitable for use with the present invention, see U.S. Pat. No. 6,911,132, entitled, “Apparatus for Manipulating Droplets by Electrowetting-Based Techniques,” issued on Jun. 28, 2005 to Pamula et al.; U.S. patent application Ser. No. 11/343,284, entitled, “Apparatuses and Methods for Manipulating Droplets on a Printed Circuit Board,” filed on filed on Jan. 30, 2006; U.S. Pat. Nos. 6,773,566, entitled, “Electrostatic Actuators for Microfluidics and Methods for Using Same,” issued on Aug. 10, 2004 and 6,565,727, entitled, “Actuators for Microfluidics Without Moving Parts,” issued on Jan. 24, 2000, both to Shenderov et al.; Pollack et al., International Patent Application No. PCT/US 06/47486, entitled, “Droplet-Based Biochemistry,” filed on Dec. 11, 2006, the disclosures of which are incorporated herein by reference. As described above, the droplet actuators include a droplet operations surface on which droplet operations are conducted. The droplet actuators also include electrodes configured for conducting droplet operations.

The droplet operations electrodes are often described here as being associated with the droplet operations surfaces, but it should be appreciated that they may be associated with any substrate of the droplet actuator, including the top and/or bottom substrates, as well as substrates which are intermediate to the top and bottom substrates, such as side walls or sealants coupling the top and bottom substrates. Further, in the various embodiments described, the top substrate may or may not be present. Various embodiments are described as using capillary forces, surface tension forces pressure sources to cause fluid to flow. It will be appreciated that in each of these embodiments any combination of capillary forces, surface tension forces, pressure sources (positive or negative) and/or other forces may be employed. Further, throughout the disclosure, the droplet actuator is typically described as having top and bottom substrates, but it will be appreciated that in embodiments that don't specifically require the droplet to be constrained between two substrates for operability, a single substrate will suffice. In embodiments that include a reservoir separated from the droplet operations surface by a reservoir wall, liquid may be introduced into the reservoir by a fluid path established in the top plate, the bottom plate and/or a side of the droplet actuator between the top and bottom plates. In addition to the various droplet dispensing protocols described herein, it should be noted that in each embodiment, a droplet may be dispensed by activating one or more of the reservoir electrodes and two or more droplet operations electrodes followed by deactivating a droplet operations electrode that is intermediate between the terminal activated droplet operations electrode and the one or more reservoir electrodes. With reference to the examples described herein, in various embodiments, 2, 3, 4, 5 or more droplet operations electrodes may be activated, followed by deactivation of an intermediate one of these droplet operations electrode to form a droplet on the terminal activated electrode or electrodes. Further, in the various embodiments described herein, a first droplet operations electrode may be adjacent to, partially embedded in or completely embedded in a reservoir electrode.

### 7.4 Fluids

For examples of fluids that may be subjected to droplet operations using the approach of the invention, see the patents listed in section 7.3, especially International Patent Application No. PCT/US 06/47486, entitled, “Droplet-Based Biochemistry,” filed on Dec. 11, 2006. In some embodiments, the fluid includes a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, fluidized tissues, fluidized organisms, biological swabs and biological washes. In some embodiment, the fluid includes a reagent, such as water, deionized water, saline solutions, acidic solutions, basic solutions, detergent solutions and/or buffers. In some embodiments, the fluid includes a reagent, such as a reagent for a biochemical protocol, such as a nucleic acid amplification protocol, an affinity-based assay protocol, a sequencing protocol, and/or a protocol for analyses of biological fluids.

7.5 Filler Fluids

The gap is typically filled with a filler fluid. The filler fluid may, for example, be a low-viscosity oil, such as silicone oil. Other examples of filler fluids are provided in International Patent Application No. PCT/US 06/47486, entitled, “Droplet-Based Biochemistry,” filed on Dec. 11, 2006.

### 7.6 Example Method of High-Throughput Droplet Dispensing

One example approach for providing a high-throughput droplet dispensing operation in a droplet actuator may include, but is not limited to, the steps of (1) providing an array of individually-controlled electrodes in the path of a liquid from which droplets to be subjected to droplet operations may be formed, such as shown in FIGS. 2 and 3; (2) providing, under a certain pressure, a volume of liquid that substantially covers the array of individually-controlled electrodes, such as shown in FIGS. 2 and 3; (3) activating certain individually-controlled electrodes, such as every other individually-controlled electrode; (4) reducing the pressure in order to cause the volume of liquid to retract starting from one end of the array of individually-controlled electrodes; and (5) forming a droplet on certain activated electrodes, such as every other electrode, in the wake of the retracting fluid, such as shown in FIGS. 2 and 3.

### 8 CONCLUDING REMARKS

The foregoing detailed description of embodiments refers to the accompanying drawings, which illustrate specific embodiments of the invention. Other embodiments having different structures and operations do not depart from the scope of the present invention.

This specification is divided into sections for the convenience of the reader only. Headings should not be construed as limiting of the scope of the invention.

It will be understood that various details of the present invention may be changed without departing from the scope of the present invention. Furthermore, the foregoing description is for the purpose of illustration only, and not for the purpose of limitation, as the present invention is defined by the claims as set forth hereinafter.

1. (canceled)

2. (canceled)

3. (canceled)

4. A method of forming multiple droplets on a droplet actuator, the method comprising:
(a) providing a droplet actuator comprising a base substrate comprising droplet operation electrodes configured for conducting one or more droplet operations;
(b) conducting the following steps in any order to provide fluid on one or more activated electrodes:
(i) flowing fluid onto at least a portion of the droplet operation electrodes; and
(ii) activating one or more of the droplet operation electrodes; and
(c) draining fluid from around the activated electrodes, leaving droplets on the activated droplet operation electrodes.

5. The method of claim 4, further comprising:
(a) providing the droplet operation electrodes in a channel on the base substrate; and
(b) using an external pressure source for flowing fluid into and retracting fluid from the channel.

6. The method of claim 4, further comprising:
(a) providing larger droplet transport electrodes alongside the droplet operation electrodes; and
(b) using the droplet transport electrodes for flowing fluid into and retracting fluid from the channel.

7. The method of claim 4 wherein the fluid comprises beads.

8. The method of claim 4 wherein the fluid comprises biological cells.

9. (canceled)

10. (canceled)

11. (canceled)

12. (canceled)

13. (canceled)

14. (canceled)

15. (canceled)

16. (canceled)

17. A method of dispensing one or more sub-droplets from a droplet on a droplet actuator, the method comprising:
(a) providing a droplet actuator comprising:
(i) a base substrate comprising electrodes configured for conducting droplet operations; and
(ii) a top substrate separated from the base substrate to form a gap, the top plate comprising:
(1) a reservoir; and
(2) an opening forming a fluid path from the reservoir into the gap;
wherein the reservoir opening is arranged such that when a fluid is provided in the reservoir, the fluid is brought into proximity to a first electrode, which first electrode is adjacent to a second electrode;
(b) causing the first and second electrodes to be activated, thereby causing fluid to flow from the reservoir onto the first and second electrodes; and
(c) deactivating the first electrode, causing a droplet to form on the second electrode and causing the remaining fluid to return substantially to the reservoir.

18. The method of claim 17 wherein the fluid comprises beads.

19. The method of claim 17 wherein the fluid comprises biological cells.

20. A method of dispensing one or more sub-droplets from a droplet on a droplet actuator, the method comprising:
(a) providing a droplet actuator comprising:
(i) a base substrate comprising:
(1) droplet operation electrodes configured for conducting droplet operations; and
(2) a recessed reservoir region configured for holding a droplet in proximity to one or more of the electrodes; and
(ii) a top substrate separated from the base substrate to form a gap;
(b) causing a first electrode adjacent to the recessed reservoir region and a second electrode adjacent to the first electrode to be activated, thereby causing fluid to flow from the reservoir onto the first and second electrodes; and
(c) deactivating the first electrode, causing a droplet to form on the second electrode and causing the remaining fluid to return substantially to the recessed reservoir region.

21. The method of claim 20 wherein the fluid comprises beads.

22. The method of claim 20 wherein the fluid comprises biological cells.

23. (canceled)

24. (canceled)

25. (canceled)

26. (canceled)

27. (canceled)

28. A droplet actuator comprising:
(a) a base substrate comprising:
(i) droplet operation electrodes configured for conducting droplet operations; and
(ii) a recessed reservoir region configured for holding a droplet in proximity to one or more of the droplet operation electrodes; and
(b) a top substrate separated from the base substrate to form a gap.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. A method of manipulating droplets on a droplet actuator, the method comprising:
(a) providing a droplet actuator comprising:
(i) a structure comprising an opening in fluid connection with a plurality of other openings;
(ii) a plurality of fluid reservoirs respectively in fluid communication with each of the other openings;
(iii) a plurality of electrodes in respective fluid communication with the fluid reservoirs; and
(iv) a plurality of flow paths through the opening, the other openings, the reservoirs and the electrodes; and
(b) flowing fluid through the plurality of flow paths.

84. The method of claim 83 further comprising serially flowing fluid through the plurality of the flow paths.

85. The method of claim 83 further comprising flowing fluid through the plurality of the flow paths in parallel.

86. A method of manipulating a droplet on a droplet actuator, the method comprising:
(a) supplying a droplet to a reservoir electrode comprising an electrode embedded in the reservoir electrode;
(b) selectively activating the embedded electrode so as to retain a portion of the droplet proximate the embedded electrode; and
(c) evacuating another portion of the droplet from the reservoir electrode.

87. The method of claim 86 further comprising another electrode embedded in the reservoir electrode, wherein the other embedded electrode is configured to retain another portion of the droplet while the other portion is evacuated.

88. The method of claim 86 wherein the droplet comprises beads.

89. The method of claim 86 wherein the droplet comprises biological cells.

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. A method of manipulating a droplet comprising magnetic beads within a droplet actuator, the method comprising:
(a) providing a droplet actuator comprising:
(i) a plurality of transport electrodes configured to transport the droplet; and
(ii) a magnetic field present at a portion of the plurality of transport electrodes; and
(b) positioning a magnetic shielding material in the droplet actuator to selectively minimize the magnetic field.

109. The method of claim 108 wherein positioning the magnetic shielding material further comprises using Mu metal.

110. The method of claim 108 wherein positioning the magnetic shielding material further comprises using nickel and iron.

111. The method of claim 108 further comprising arranging a magnet producing the magnetic field and the plurality of transport electrodes into a lane.

112. The method of claim 111 further comprising positioning a plurality of lanes in the droplet actuator.

113. The method of claim 112 further comprising positioning the magnetic shielding material to minimize the affects of the magnetic fields emanating from the respective lanes.

114. (canceled)

115. (canceled)

116. (canceled)

117. (canceled)

118. (canceled)

119. A method of re-suspending particulate within a droplet in a droplet actuator, the method comprising:
(a) providing a droplet actuator comprising:
(i) a reservoir electrode configured to manipulate a droplet; and
(ii) a plurality of transport electrodes in fluid communication with the reservoir electrode;
(b) separating a slug of the droplet from the droplet on the reservoir electrode; and
(c) recombining the slug with the droplet at the reservoir electrode.

120. The method of claim 119 further comprising transporting the slug along the plurality of transport electrodes.

121. The method of claim 119 further comprising repeating steps (b) and (c).

122. The method of claim 119 wherein the particulate comprises a bead.

123. The method of claim 119 wherein the particulate comprises a biological cell.

124. A method of re-suspending particulate within a droplet in a droplet actuator, the method comprising:
(a) providing a droplet actuator comprising:
(i) a reservoir electrode configured to manipulate a droplet; and
(ii) a plurality of transport electrodes in fluid communication with the reservoir electrode; and
(b) selectively applying across the reservoir electrode a voltage from an alternating current source to agitate the droplet.

125. The method of claim 124 wherein the particulate comprises a bead.

126. The method of claim 124 wherein the particulate comprises a biological cell.

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. (canceled)

143. (canceled)

144. (canceled)

145. (canceled)

146. (canceled)

147. (canceled)

148. (canceled)

149. (canceled)

150. (canceled)

151. (canceled)

152. (canceled)

153. (canceled)

154. (canceled)

155. (canceled)

156. (canceled)

157. (canceled)

158. (canceled)

159. (canceled)

160. (canceled)

161. (canceled)

162. (canceled)

163. (canceled)

164. (canceled)

165. (canceled)

166. (canceled)

167. (canceled)

168. (canceled)

169. (canceled)

170. (canceled)

171. (canceled)

172. (canceled)

173. (canceled)
